(19) United States
(12) Patent Application Publication
(10) Pub. No.: US 2025/0207177 A1
(43) Pub. Date: Jun. 26, 2025

(54) METHODS, COMPOSITIONS AND DEVICES FOR SPREADING OF CHROMATIN FIBERS

(71) Applicant: Erasmus University Medical Center Rotterdam, Rotterdam (NL)

(72) Inventors: Nitika TANEJA, Rotterdam (NL); Vincent GAGGIOLI, Rotterdam (NL); Roland KANAAR, Rotterdam (NL)

(21) Appl. No.: 18/844,163

(22) PCT Filed: Mar. 10, 2023
(86) PCT No.: PCT/NL2023/050120

§ 371 Date: Sep. 5, 2024

(30) Foreign Application Priority Data
Mar. 10, 2022 (NL) 2031242

(51) Int. Cl. C12Q 1/6806; G01N 1/28

(52) U.S. Cl. CPC C12Q 1/6806; G01N 1/2813

(57) ABSTRACT

The present invention provides a method of spreading DNA fibers on a surface of a microscope slide for microscopic analysis, the method comprising the steps of (i) providing a cell or cellular compartment comprising DNA fibers to be analyzed; (ii) providing a microscope slide for microscopic analysis; (iii) providing a container holding an amount of a liquid lysis composition for lysing said cell or cellular compartment; (iv) adhering said cell or cellular compartment to the surface of said microscope slide; (v) transferring said slide with said cell or cellular compartment adhered thereto to said container thereby substantially submerging said slide in said lysis composition, wherein said slide is placed in said lysis composition at a predetermined angle with respect to the horizontal plane of between 45° and 90°, and wherein the adhered cell or cellular compartment is facing upward; and (vi) allowing lysis of said cell or cellular compartment adhered to said slide, and removing said lysis composition from said container at a controlled and constant rate of flow by use of a liquid pump to thereby facilitate spreading of the DNA fibers on the surface of said microscope slide.

### FIELD OF THE INVENTION

The invention is in the field of microscopic analysis of isolated DNA fibers and associated proteins, such as analysis of chromatin fibers. The invention is directed to an optimized method for spreading and producing extended chromatin fibers on microscope slides, which can be subsequently processed for microscopy, including super-resolution fluorescence microscopy. The individualized chromatin fibers on the slide can be used for a broad array research purposes, for example in the investigation of replication forks, DNA repair, transcription regulation, epigenetic marks and chromatin-associated proteins. The invention is further directed to devices and compositions for use in the method of the invention.

### BACKGROUND OF THE INVENTION

For investigation of numerous cellular processes, single molecule techniques have become increasingly important. In order to conduct experiments involving chromatin, isolation and individualization of chromatin fibers have to be performed with the greatest care. In addition, single molecule experiments require a large number of samples in order to gain statistical significance. Hence, both efficient and qualitative isolation and individualization of chromatin fibers is key to producing reliable results.

However, prior art protocols for preparing chromatin for single molecule experiments, such as protocols described in WO9841651A1, Wooten et al. (Nature Protocols 2020;15(3):1188-1208), Blower et al. (Dev Cell 2002; 2(3):319-330) and Dunleavy (Epigenesys Protocol 55. Preparation of extended chromatin fibers from human tissue culture cells (Prot55), 18 Apr. 2012), involve a number of disadvantages. Lysis of cells, from which chromatin is isolated, is notoriously inefficient, leading to a low chromatin yield. Also, isolated chromatin fibers often are entangled, which renders them useless in single molecule experiments. For those reasons, it has been incredibly difficult to obtain useful images using conventional or super-resolution fluorescent microscopy setups. Until now, only electron microscopy (EM) has been able to provide detailed images of sub-structures and protein interactions of chromatin. However, EM completely disables dynamic processes and precludes the observation of the chromatin landscape and proteins associated with DNA. Likewise, a technique known as DNA combing which allows observation of individualized DNA fibers, is expensive and preclude the observation of proteins associated to DNA as one of the essential steps of the techniques is to remove the proteins from the samples. Also, EM and DNA combing require a substantial amount of equipment and expertise, and can therefore be performed in a limited number of laboratories.

Alternatively to DNA combing, manual extraction and spreading of DNA can be done to cut costs and reduce the need for specialized equipment. Disappointingly, manual extraction and DNA spreading is very labor-intensive and leads to highly variable results. Also, when spreading the DNA on microscope slides, known techniques lack control over the direction and velocity of the flow, resulting in non-reproducible DNA fragment lengths and amounts, as well as variability in the extent to which the isolated DNA duplexes are stretched on the slide. Existing methods cannot be scaled up, which means DNA extraction and spreading is very time consuming when attempting to acquire data from a large number of stretched DNA duplexes and/or fibers.

There is a need in the art for methods that allow for isolation of stretched chromatin fibers at single molecule level, preferably at large scale, that are reproducible, and that provide stretched chromatin on surfaces for microscopic evaluation of cellular processes, such as relating to DNA replication, DNA repair, protein interaction, chromatin landscape, chromatin dynamics, post-translational modifications, transcription regulation and DNA-related drug screening.

### SUMMARY OF THE INVENTION

The present inventors herein disclose a method for chromatin stretching that allows for a reproducible and highly efficient isolation of single chromatin fibers on a microscope slide. The slides on which said chromatin is stretched, can directly be used in a wide variety of imaging techniques, such as many forms of fluorescence imaging, including super-resolution microscopy. The disclosed method is faster and more efficient compared to known techniques, allows for large scale (semi)automated processing of chromatin stretching on slide surfaces. The method results in stretched chromatin fibers that are intact and show a high degree of individualization. The method, for instance, allows for the direct examination of proteins in association with a DNA replication fork at the single molecule level, both in a qualitative and quantitative manner. The method is preferably used in combination with device of the invention as disclosed herein. The device minimizes the need for manual steps, thereby leading to a reduction in variability in both the quantity and quality of the stretched chromatin fibers.

The inventors further disclose compositions that can beneficially be used in a method for chromatin stretching according to the invention.

Finally, the inventors disclose a device that allows for an even more reproducible and efficient method for DNA extraction and individualization.

In a first aspect, the present invention provides a method of spreading DNA fibers on a surface of a microscope slide for microscopic analysis, the method comprising the steps of:
(i) providing a cell or cellular compartment comprising DNA fibers to be analyzed;
(ii) providing a microscope slide for microscopic analysis;
(iii) providing a container holding an amount of a liquid lysis composition for lysing said cell or cellular compartment;
(iv) adhering said cell or cellular compartment to the surface of said microscope slide;
(v) transferring said slide with said cell or cellular compartment adhered thereto to said container thereby substantially submerging said slide in said lysis composition, wherein said slide is placed in said lysis composition at a predetermined angle with respect to the horizontal plane of between 45° and 90°, and wherein the adhered cell or cellular compartment is facing upward;
(vi) allowing lysis of said cell or cellular compartment adhered to said slide, and removing said lysis composition from said container at a controlled and constant rate of flow by use of a liquid pump to thereby facilitate spreading of the DNA fibers on the surface of said microscope slide, and
(vii) optionally fixing and/or staining the thus spread DNA fibers prior to microscopic analysis.

In a preferred embodiment of a method according to the present invention the cellular compartment is a cell nucleus. In such preferred embodiments, step (i) of a method of the invention preferably comprises the steps of providing a cell comprising DNA fibers to be analyzed, solubilizing the cell membrane and extracting the intact cell nucleus from said cell. In such preferred embodiments, step (iv) of a method of the invention preferably comprises the step of adhering said intact cell nucleus to the surface of said microscope slide.

In another preferred embodiment of a method according to the present invention, the lysis composition is an aqueous buffer comprising as a buffering agent 2-(-N-morpholino)ethanesulfonic acid (MES), preferably at a pH in the range of 6.0-6.5. MES is preferably present in such a lysis composition in an amount of between 40-60 mM, preferably at about 50 mM. The present inventors have found that a MES buffer provides for superior lysis and spreading relative to the use of other buffers, such as a tris(hydroxymethyl)aminomethane (Tris) buffer.

In another preferred embodiment of a method according to the present invention, the lysis composition is an aqueous buffer comprising a nonionic detergent, preferably Triton X-100, preferably at 2 wt. %.

The present inventors have further found that the amount of an ionic salt, preferably NaCl, is preferably lower than 500 mM, preferably around 100 mM.

The inventors have further found that NaCl can be omitted entirely from the lysis composition. The present invention therefore provides in aspects a lysis composition that is referred to herein as hypotonic, meaning that an ionic salt, preferably NaCl, may be absent from the lysis composition. The inventors have further found that the presence of urea for sufficient lysis of whole cells is not required if the lysis composition (in addition to the indicated preferred amount of MES buffer and Triton X-100) comprises divalent cation-chelating agents, preferably ethylenediaminetetraacetic acid (EDTA) and ethylene glycol tetraacetic acid (EGTA), preferably at 0.1 mM each. Hence, in a preferred embodiment of a lysis composition according to the present invention, the lysis composition contains about 50 mM MES (pH 6.5), about 0.1 mM EDTA, about 0.1 mM EGTA, and about 2 wt. % Triton X-100. It was found that this lysis composition results in very good lysis of cells, high quality of spreading, high quality of immunofluorescent staining, and a high quantity of analyzable fibers.

Still further improved lysis of cells, and a concomitant increase in the quantity of analyzable fibers may be obtained by prior extraction of the nuclei from the cells, and adhering these cell compartments to the microscope slide. When using extracted nuclei, the inventors found that the use of a reducing agent, preferably dithiothreitol (DTT), preferably at 1 mM, is preferred and provides for various benefits. A highly preferred lysis composition according to the present invention is hypotonic, and preferably contains about 50 mM MES (pH 6.5), about 0.1 mM EDTA, about 0.1 mM EGTA, about 1 mM DTT, and about 2 wt. % Triton X-100. This composition ensures high lysis efficiency with a concomitant high quantity of analyzable fibers on the slide, a high quality of the DNA-chromatin spreading, and a high quality of immunofluorescence staining. Preferably, a lysis and spreading composition in aspects of this invention consists of 50 mM MES (pH 6.5), 0.1 mM EDTA, 0.1 mM EGTA, 1 mM DTT, and 2 wt. % Triton X-100

Since immunofluorescent staining is particularly aimed at visualizing the proteins associated with the DNA, the DNA fiber in methods of this invention is preferably a chromatin fiber.

The spreading of the DNA fibers in methods of this invention is so beneficial, particularly in combination with the improved flow of the lysis composition over the slide surface due to the use of a device as described herein, that it results in a high quality of spreading. This high quality of spreading is also attained for protein-free DNA fibers. Hence, the present invention also envisions the analysis of protein-free DNA fibers in methods of this invention. When investigating protein-free DNA fibers, spreading may occur using a lysis composition that preferably comprises sodium dodecyl sulphate (SDS), preferably at about 1 wt. %. Buffers other than MES buffers, such as Tris buffers, may also be used in such aspects, but MES is highly preferred, most preferably at about pH 6.5.

It is a preferred embodiment in methods of this invention that the staining of the DNA fibers comprises immunofluorescent staining.

In some embodiments of this invention it is possible that step (i) further involves (or is preceded by) incubation with a nucleoside analog, and allowing incorporation in said nucleoside analog in said DNA fiber.

The DNA fiber spread on the microscope slide using methods of this invention may be analyzed by fluorescence microscopy, preferably super resolution microscopy, such as Structured Illumination Microscopy (SIM) or Stimulated Emission Depletion (STED) microscopy or derivates thereof.

In another aspect, the present invention provides a microscope slide provided with DNA fibers spread by the method according to the invention as described above.

In yet another aspect, the present invention provides a device for spreading DNA fibers on a microscope slide, preferably said device being arranged for use in a method according to the invention as described above. In a preferred embodiment, the device of the present invention comprises a liquid container for holding an amount of liquid (preferably a lysis and spreading composition according to the present invention), wherein the liquid container is arranged for holding at least one slide therein in a manner in which said at least one slide is substantially submerged in said liquid held in the liquid container during use of the device, wherein the device is arranged to hold the at least one slide at a predetermined angle with respect to the horizontal plane, said angle being between 10° and 170°, preferably between 20° and 160°, more preferably between 30° and 150°, yet more preferably between 40° and 140°, wherein the device further comprises a pump for pumping the liquid out of the liquid container.

In a preferred embodiment of the device of the present invention, the pump is a peristaltic pump. In further preferred embodiments, the device, and in particular the pump, is arranged for pumping the liquid out of the liquid container at a substantially constant flow rate, preferably wherein the liquid held in the liquid container during use of the device is about 30-50 ml, and preferably wherein said flow rate is between 1 ml/min and 100 ml/min, more preferably between 2 ml/min and 50 ml/min, yet more preferably between 5 ml/min and 25 ml/min, such as for instance a flow rate of about 10 ml/min or about 15 ml/min. In a preferred embodiment, the liquid is pumped out of the liquid container through an opening in or at or near the bottom of the container.

In another preferred embodiment of the device of the present invention, the liquid container is arranged for holding a multiple number of slides therein, preferably in a manner in which multiple ones, and preferably all, of said multiple number of slides are held substantially parallel with each other, more preferably wherein the slides are substantially uniformly offset such that an offset distance between two adjacent slides will be substantially equal to the offset distance between any two other adjacent slides, yet more preferably wherein said offset distance is between 4 mm to 12 mm, preferably 8 mm to 12 mm, most preferably about 10 mm.

In still another preferred embodiment of the device of the present invention, the device comprises a base portion and at least one container holder, and preferably multiple container holders, for retaining at least the one liquid container therein, preferably wherein the at least one container holder is adjustably mounted to the base portion such that during use of the device the angle of at least one slide held in the liquid container which in turn is held in the container holder can be adjusted.

In yet another preferred embodiment of the device of the present invention, the multiple number of slides in a device of the invention are held in said liquid container in a removable insert comprising slits for holding individual slides at predetermined offset.

In another aspect, the present invention provides for the use of the device according to the present invention, preferably in a method according to the present invention as described above.

In still another aspect, the present invention provides for a system for the preparation of a microscope slide comprising spread chromatin fibers on the surface of said slide for microscopic analysis, the system comprising (a) an aqueous lysis composition consisting of 50 mM MES (pH 6.5), 0.1 mM EDTA, 0.1 mM EGTA, 1 mM DTT, and 2 wt. % Triton X-100, and (b) a device according to the present invention as described above. A system of the invention preferably further comprises (an instruction for) performing the method of spreading DNA fibers on a surface of a microscope slide for microscopic analysis as described above.

### DESCRIPTION OF THE DRAWINGS

By way of non-limiting examples only, embodiments of the present invention will now be described with reference to the accompanying figures. It is noted that the figures show merely a preferred embodiment according to aspects of the invention. In the figures, the same or similar reference signs or numbers refer to equal or corresponding parts.

FIG. 1. Schematic perspective view of a first embodiment of a device for isolating nucleic acids on a slide according to an aspect of the invention.

FIG. 2. Further schematic perspective view of the device of FIG. 1 in which some components of the device have been omitted. FIG. 3. Further schematic perspective view of the device of FIG. 1. In this figure, it is demonstrated that the angle of the apparatus can for example be adjusted between 0° and 45° with respect to the vertical.

FIG. 4. Stained replication forks imaged using a super-resolution SIM microscope. In the left panels, an active replication fork has been generated using a method of the invention. In the right panels, a stalled, hydroxy urea (HU)-treated replication fork has been generated using a method of the invention. DNA has been stained using 4′,6-diamidino-2-phenylindole (DAPI); newly synthesized DNA has been stained using 5-ethynyl-2′-deoxyuridine (EdU)-interacting fluorophores, and Proliferating cell nuclear antigen (PCNA) and Replication protein A (RPA) have been stained by immunofluorescence using primary and secondary antibodies for detecting the specific proteins.

FIG. 5. Influence of a method of the invention on quantification of protein intensity. A. From left to right, DNA; Proliferating cell nuclear antigen (PCNA); nascent DNA; composite of nascent DNA and PCNA; and composite of DNA nascent DNA and PCNA are shown, as prepared using a prior art protocol for preparation of extended chromatin fibers as referenced above. A maximum of 10 fibers was found on one microscope slide. B. Preparation of fibers using the prior art protocol induces high variability in quantification of PCNA intensity. This is due to inefficient separation of fibers. C. From left to right, DNA; Proliferating cell nuclear antigen (PCNA); nascent DNA; composite of nascent DNA and PCNA; and composite of DNA nascent DNA and PCNA are shown, as prepared using the method of the invention. A maximum of 500 fibers was found on one microscope slide. D. Preparation of fibers using the method of the invention greatly reduces variability in quantification of PCNA intensity within slides as well as between slides. This is due to a better separation of fibers compared to the prior art protocol.

FIG. 6. Quantification of Replication protein A (RPA). The fibers which were subjected to Quantification of RPA were prepared using a method of the invention.

FIG. 7. Observation of histone modifications and quantitative analysis. A. From left to right, DNA; histone variant H3K9me1; nascent DNA; composite of nascent DNA and H3K9me1; and composite of DNA nascent DNA and H3K9me1 are shown, as prepared using the method of the invention. B. A method of the invention allowed for accurate quantification of H3K9me1 on DNA.

FIG. 8. Comparison between manual DNA fiber spreading and automatic DNA fiber spreading using the method of the invention. The result of staining after manual DNA fiber spreading is demonstrated in the top panels; that of staining after automatic DNA fiber spreading using the method of the invention is shown in the bottom panels. DNA was marked by immuno fluorescence using a mouse anti-DNA (primary antibody) and goat anti-mouse coupled to a fluorophore (secondary antibody); newly synthesized DNA was marked using fluorophores that interact with nucleotide analogs iododeoxyuridine (IdU) and chlorodeoxyuridine (CldU). Compared to conventional manual DNA fiber spreading, the directionality of fibers is much improved, as the fibers are straighter. The result of a method of the invention is similar to the highly expensive DNA combing technique. Manually spread fibers get fragmented and appear in all directions, which makes analysis difficult and contributes to high variability of the signal. The method of the invention also improved individualization of fibers, as well as the staining of DNA.

### DETAILED DESCRIPTION OF THE INVENTION

### Definitions

The terms ‘isolation’ and ‘isolating’, as used herein, include reference to the extraction of a compound from a cell, with the aim of keeping said compound. In particular, in the context of the invention, isolation refers to the extraction of nucleic acids from a cell, optionally together with associated proteins. Isolation can be performed on nucleic acids from any organisms, including eukaryotes, archaea and bacteria. As nucleic acids are contained by a membrane, such as a nuclear membrane, mitochondrial membranes or cytosolic membrane, isolation implies that the membranes that contain the nucleic acids of interest are disrupted to an extent that allows for the nucleic acids to leave the compartment in which they were contained. Isolation of nucleic acids from a cell may thus include destruction of the cell in order to release nucleic acids and/or compartments holding nucleic acids. In the context of eukaryotes, isolation explicitly also implies disruption of the nucleus. Preferably, when isolating nucleic acids, only nucleic acids and optionally associated molecules are isolated; other cellular components are discarded as a result of the isolation process.

The term ‘individual separation’, as used herein, includes reference to the dissociation and/or untangling of individual molecules or complexes from molecules of the same or similar kind, optionally while still together with molecules that are associated with said molecule or complex. Said term can interchangeably be used with the term ‘individualization’. In particular, individual separation refers to individual separation of nucleic acids. When nucleic acids are individually separated, this process results in nucleic acids that are to a lesser extent than before non-covalently connected to other nucleic acids. The individual separation of nucleic acids does not necessarily lead to full individualization; a sample after individual separation may still comprise non-individualized nucleic acids. When used in the context of DNA, individual separation of DNA duplexes is meant; individualization explicitly does not refer to the melting of a DNA duplex, whereby two single stranded DNA molecules are separated. Preferably, proteins and small molecules that were associated with the nucleic acid before individual separation are still present after individual separation. For example, histones are preferably not removed from DNA during individual separation. Individual separation must occur during in order to prepare extended chromatin fibers.

The term ‘nucleic acid’, as used herein, includes reference to biopolymers composed of nucleotides, such as DNA and RNA. Preferably, nucleic acid refers to DNA.

The term ‘DNA fiber’, as used herein, refers to a deoxyribonucleic acid (DNA) molecule. The term includes reference to a DNA molecule in complex with proteins and optionally other nucleic acid (e.g. RNA) molecules, and to DNA molecules which are substantially free of protein or other cellular components. The term includes reference to double stranded DNA as well as a single stranded DNA. DNA/protein complexes covered by the term ‘DNA fiber’ in particular include chromatin, including nucleosomes. Certain aspects of the present invention are particularly suitable for chromatin stretching. However, stretching of DNA fibers substantially free of protein or other cellular components is also envisioned herein.

The term ‘chromatin’, as used herein, includes reference to a complex of DNA and DNA-associated proteins, such as histones. Chromatin may refer to multiple forms and stages of protein-complexed DNA, including a beads-on-a-string conformation, euchromatin, heterochromatin, mitotic and meiotic chromatin. The term ‘nascent chromatin’, as used herein, includes reference to chromatin, more particularly to forms in which the DNA component of chromatin is newly synthesized by the cell. The term ‘chromatin fiber’, as used herein, preferably refers to the complex of DNA and protein, such as found in eukaryotic cells, wherein DNA, histones and optionally RNA and DNA polymerases, RNA other proteins are arranged in a complex, for instance in the form of a fiber having a width of approximately 30 nm. This 30 nm complex is formed when nucleosomes fold together. However, the term “fiber” is not particularly limited herein, and includes reference to all structural forms of the chromatin molecule comprising a complex of DNA and protein.

The term ‘slide’, as used herein, includes reference to an object with an approximately flat top surface whereon biological material may be placed, and that is compatible with an imaging technique and/or an imaging device, such as in particular an optical microscope, more in particular for instance a fluorescence microscope, including conventional widefield or confocal microscopes, and in particular using fluorescence microscopic techniques of super-resolution, such as SIM. An exemplary slide in the context of the invention is a microscope slide, which can be formed as a relatively thin piece of glass comprising two flat surfaces and generally four edges (if square or rectangular) or one edge (if round or oval), and which can be used in a wide array of microscopy techniques, for example in super-resolution microscopy techniques. Suitable slides can for example be made of glass, such as silicate glass, soda-lime glass, borosilicate glass, lead glass, aluminosilicate glass, lead glass; or of quartz or fused quartz. Slide surfaces may be coated or otherwise treated, for example in order to improve adhesion of cells to a surface of a slide. A slide in aspects of this invention preferably comprises at least one application location for applying the cell or cellular compartment (or a sample of cells or cell compartments) for adhesion to the slide surface. This at least one application location may be centered on the surface of the slide. Preferably, the application location on the slide is situated above the liquid outlet port or outlets when the slide is placed in the container.

The terms ‘adhesion’, and ‘surface adhesion’, as used herein, include reference to the process by which cells or cell components, such as DNA, attach to a surface, in particular the surface of a slide as used in a method of the invention, such as a microscope slide, preferably an (sample) application location of a slide. Surface adhesion may happen under conditions that allow for such adhesion to occur, which conditions may require specific lengths of time, temperature, pH, salt or other conditions, which conditions can easily be optimized by the person of average skill in the art. Additionally, coating of a surface may improve surface adhesion of cells. Such coating may for instance include an acrylate or silan coating of the surface.

The term ‘solubilizing’, as used herein, refers to the solubilization of the cytoplasmic cell membrane, resulting in lysis of a cell and release of cellular components therefrom. Organelles, cellular compartments, nucleic acids, proteins and other cellular content may be released from the cell. Solubilization may be achieved using chemicals such as detergents, which act by disintegrating the lipid bilayer of the cytoplasmic membrane.

The term ‘lysis’, as used herein, includes reference to the disruption of a membrane, allowing the components of the compartment that was formed by said membrane to release to outside the compartment. Lysis may for example be caused, or partially be caused, by osmotic imbalance, chemicals, acoustic waves or mechanical rupture of the membrane. Preferably, lysis is achieved by use of a lysis composition as described herein.

The term ‘lysis composition’, as used in aspects of this invention, refers in particular to the aqueous liquid in which the slides with adhered cells or cell compartments are submerged, and which may also be referred to as the spreading composition that causes spreading of the DNA fiber on the surface of the slide due to drag forces imparted on the liberated DNA as a result of laminar fluid flow draining away from the containers holding the lysis composition and slides with adhered cells or cell compartments submerged therein.

The term ‘membrane’, as used herein, includes reference to a composition of lipids—mainly phospholipids—and optionally glycolipids, membrane proteins and sterols, that makes up the boundary of cellular compartments. Said lipids form a lipid bilayer that prevents larger molecules from passively diffusing from one side of the membrane to the other. Examples of cellular compartments that comprise a membrane are the cytoplasmic membrane, the nucleus, the Golgi system, the endoplasmic reticulum, the mitochondria, the vacuole, the chloroplast, the peroxisomes and the lysosomes. The term ‘cytoplasmic membrane’, as used herein, also referred to as ‘cell membrane’ and ‘plasma membrane’, includes reference to the membrane that separates the interior of a cell from the extracellular space. The cytoplasmic membrane contains all components of the cell while facilitating active and passive transport between the intracellular and extracellular space. Cell cytoplasmic membranes are distinct from other membranes, such as membranes of the mitochondria, chloroplasts, nucleus, endoplasmic reticulum and Golgi apparatus in both function and composition.

The term ‘nucleus’, and its plural ‘nuclei’, as used herein, includes reference to the compartment of the eukaryotic cell wherein the cell's genomic material is located. The nucleus is formed by the nuclear membrane. Most eukaryotic cells possess a nucleus.

The term ‘fixing’ or ‘fixating’, as used herein, includes reference to the chemical crosslinking of cell components using a fixative.

The term ‘fixative’, as used herein, includes reference to a compound that can be beneficially used for the fixation of biological material. Examples of fixatives are aldehyde fixatives, such as formaldehyde, glutaraldehyde and acrolein (also referred to as propenal), or mixtures thereof, oxidizing fixatives such as osmium tetroxide, alcohols, such as ethanol or methanol, acetone, mercurial fixative such as Zenker's fixative and HOPE (Hepes-glutamic acid buffer-mediated organic solvent protection effect) fixative. The terms “formaline” and “paraformaldehyde” may be used interchangeably with the term “formaldehyde”, although strictly speaking formaline refers to a 37% w/v or 40% v/v solution of the water-soluble gas formaldehyde in water together with up to 15% v/v methanol, and paraformaldehyde refers to higher polymers of formaldehyde that are poorly soluble in water. A fixative may also refer to a mixture comprising multiple fixatives.

The term ‘flow’, as used herein, includes reference to motion of liquid along a surface, in particular along the surface as used in a method of the invention. Said liquid may for example be a composition, such as a composition of the invention. The term ‘controlled flow’, as used herein, includes reference to a flow of which the rate can be adjusted and set to a specific value. A flow can for example be controlled by the use of a pump. A flow wherein the rate is controlled by gravity, for example by letting liquid flow out of a container via a hole in said container wherein said flow is essentially solely a result of gravitational force, is not regarded as a controlled flow. The term ‘constant flow’, as used herein, includes reference to a flow that has an approximately non-fluctuating rate. A flow wherein the rate is controlled by gravity, for example by letting liquid flow out of a container via a hole in said container wherein said flow is essentially solely a result of gravitational force, is not regarded as a constant flow, because said flow rate decreases as the liquid level in the container decreases.

The term ‘longitudinal direction’, as used herein, includes reference to a direction parallel to the surface along which an object moves, preferably also parallel to the longest edge. For example, an object moving along a rectangular microscope slide that is positioned on its short edge in longitudinal direction moves towards the short edge of the microscope slide, in a direction that forms a 90° C. angle with said short edge.

The term ‘spreading’, as used herein, includes reference to the longitudinal migration of a nucleic acid or a part thereof, thereby roughly aligning with the longitudinal axis of the surface on which they are spread. Ideally, nucleic acids remain fixed to the surface on one end while the remaining part stretches away from said end.

The term ‘reducing agent’, as used herein, includes reference to a compound that may react with an electron recipient by transferring an electron to said recipient. Non-limiting examples of reducing agents are lithium aluminium hydride, Red-Al, hydrogen, sodium amalgam, zinc amalgam, diborane, sodium borohydride, sulfur dioxide, dithionates, thiosulfates, iodides, hydrogen peroxide, hydrazine, diisobutylaluminium hydride, oxalic acid, formic acid, ascorbic acid, reducing sugars, phosphites, hypophosphites, phosphorous acid, DTT, carbon monoxide, cyanides, carbon and tris-2-carboxyethylphosphine hydrochloride (TCEP).

The term ‘nonionic detergent’, as used herein, includes reference to detergents with a hydrophilic head group that is uncharged. Non-limiting examples of nonionic detergents are Triton X-100 (also referred to as polyethylene glycol mono(p-(1,1,3,3-tetramethyl-butyl)phenyl)ether), Triton X-114, Nonidet P-40 substitute, Tween 80 (also referred to as polyoxyethylene (20) sorbitan monooleate), octyl beta glucoside, MEGA 8, MEGA 9, MEGA 10, Big CHAP, deoxy Big CHAP, Brij 35 and Brij 58.

The term ‘buffering agent’, as used herein, includes reference to a compound which in an aqueous solution can be used to stabilize pH to a certain extent. Non-limiting examples of buffering agents are citric acid, acetic acid, potassium dihydrogen phosphate, sodium dihydrogen phosphate, dipotassium hydrogen phosphate, disodium hydrogen phosphate, CHES (N-cyclohexyl-2-aminoethanesulfonic acid), borate, TAPS ([tris(hydroxymethyl)methylamino]propanesulfonic acid), bicine (2-(bis(2-hydroxyethyl)amino)acetic acid), Tris (a.k.a. tris base), Tricine (N-[tris(hydroxymethyl)methyl]glycine), TAPSO (3-[N-tris(hydroxymethyl)methylamino]-2-hydroxypropanesulfonic acid), HEPES (4-(2-hydroxyethyl)-1-piperazineethanesulfonic acid), TES (2-[[1,3-dihydroxy-2-(hydroxymethyl)propan-2-yl]amino]ethanesulfonic acid), MOPS (3-(N-morpholino)propanesulfonic acid, PIPES (piperazine-N,N′-bis(2-ethanesulfonic acid)), Cacodylate (dimethylarsenic acid) and MES. The term ‘phosphate-comprising buffering agent’, as used herein, includes reference to a buffering agent that comprises phosphate ions. Non-limiting examples of phosphate-comprising buffering agents are potassium dihydrogen phosphate, sodium dihydrogen phosphate, dipotassium hydrogen phosphate and disodium hydrogen phosphate.

The term ‘ionic salt’, as used herein, includes reference to a compound composed of anions and cations with no net electric charge, or the cations and anions thereof. Said term can interchangeably be used with the term ‘salt’.

The term ‘resuspending’, as used herein, includes reference to bringing a cell into suspension in a buffer that is suitable therefor. The resulting suspension comprises the resuspended cell. Preferably, resuspended cells are not attached to other cells.

The term ‘hypotonic’, as used herein, includes reference to solutions of which the tonicity is lower than that of cells in said solution, or of cells that are intended to be contacted with said solution. In particular, the tonicity of a hypotonic solution is lower than that of the cytosol of said cell, causing water to diffuse into the cell due to osmotic pressure. This may result in rupture of the cytosolic membrane and subsequent lysis.

The term ‘imaging technique’, as used herein, includes reference to techniques which allow for the visualization of objects. In the context of the invention, said objects particularly include reference to nucleic acids and optionally to proteins and other compounds associated with nucleic acids.

The term ‘light microscopy’, as used herein, includes reference to an imaging technique wherein visible light is used. Non-limiting examples of techniques that are subclasses of light microscopy, are bright field microscopy, dark field imaging, dispersion staining microscopy, phase contrast microscopy, differential interference contrast microscopy, interference reflection microscopy, fluorescence microscopy, confocal microscopy, two-photon microscopy, and combinations thereof. Light microscopy techniques are usually limited by the diffraction limit, but additional deconvolution techniques may allow for imaging below the diffraction limit.

The term ‘fluorescence microscopy’, as used herein, includes reference to a light microscopy technique wherein light from fluorescence is used instead of or in addition to conventional light sources. Fluorescent material may be introduced to the sample or may already have been present in the sample, for example due to the introduction or modification of genes that produce fluorescent proteins. Non-limiting examples of fluorescent stains that may be used in fluorescence microscopy are DAPI, Hoechst, DRAQ5, DRAQ7, phalloidin, SYBR green, fluorescein, and Alexa fluors. Non-limiting examples of fluorescent proteins that may be used in fluorescence microscopy are green fluorescent protein (GFP) mCherry, yellow fluorescent protein (YFP), mBanana, YPet, mOrange, DsRed, mRuby, cyan fluorescent protein (CFP), mEOS and mutants and variants thereof. Fluorescent stains and proteins may be linked to specific components via linkage to mediating proteins, such as antibodies. The latter technique may be referred to as immunofluorescence, and may be primary, wherein the fluorophore is located on the antibody that targets the structure of interest, or secondary, whereby the fluorophore is located on an antibody that binds an unlabeled antibody.

The term ‘super-resolution microscopy’, as used herein, includes reference to light microscopy techniques that allow for the detection of structures and components below the diffraction limit. Non-limiting examples of super-resolution microscopy techniques are photon tunneling microscopy (PTM), near-field optical random mapping (NORM) microscopy, structured illumination microscopy (SIM), spatially modulated illumination (SMI), stimulated emission depletion (STED), ground state depletion (GSD), saturated structured illumination microscopy (SSIM), spectral precision distance microscopy (SPDM), cryogenic optical localization in 3D (COLD), binding-activated localization microscopy (BALM), stochastic optical reconstruction microscopy (STORM), photo activated localization microscopy (PALM), fluorescence photo-activation localization microscopy (FPALM), points accumulation for imaging in nancoscale topography (PAINT), direct stochastic optical reconstruction microscopy (dSTORM), super-resolution optical fluctuation imaging (SOFI), omnipresent localization microscopy (OLM) and 3D light microscopical nanosizing (LIMON) microscopy. Some super-resolution microscopy techniques can be combined with fluorescence microscopy or require to be combined with fluorescence microscopy.

The term ‘labeling’, as used herein, includes reference to covalently or non-covalently attaching a detectable marker to a nucleic acid or protein, in such a way that said nucleic acid or protein can be visualized using an imaging technique. In particular, the detectable marker is a fluorophore that can be attached to a nucleic acid or protein to allow visualization using fluorescence microscopy and/or super-resolution microscopy. Said detectable marker can be organic or inorganic. Attachment can occur in any possible way, for example via antibody interaction, biotin-streptavidin coupling, intercalation, click chemistry or chemical incorporation.

The terms ‘incubation’ and ‘incubating’, as used herein, include reference to contacting a cell with a composition under conditions that allow said composition to interact with said cell. Particularly, said term refers to contacting a cell with a composition comprising a compound that during incubation is taken up by the cell.

The term ‘nucleoside analog’, as used herein, includes reference to a compound analogous to thymidine, adenosine, guanosine, uridine or cytidine. Non-limiting examples of nucleoside analogs are radioactively labeled nucleoside analogs, 5-bromodeoxyuridine (BrdU), 5-chlorodeoxyuridine (CldU), 5-iododeoxyuridine (IdU), 5-ethynyl-2′-deoxyuridine (EdU) and 5-azido-UDP (AdU). Incubation of a cell with a nucleoside analog may result in the incorporation of said nucleoside analog into the DNA of said cell. Some nucleoside analogs, such as the thymidine analog EdU, may be covalently linked to a fluorescent dye-conjugated azide via a copper [Cu(I)]-catalyzed [3+2] cycloaddition reaction. Thereby, DNA wherein EdU has been incorporated, can be visualized using fluorescent microscopy and/or super-resolution microscopy.

### Methods

The present invention now provides a method to provide stretched DNA or chromatin fibers on a surface for microscopic evaluation, wherein the quality of the separation of individual fibers is very good, wherein the yield of fibers from a cell is very high and wherein the immunochemical staining is of very high quality.

The method comprises the steps of
(i) providing a cell or cellular compartment comprising DNA fibers to be analyzed;
(ii) providing a microscope slide for microscopic analysis;
(iii) providing a container holding an amount of a liquid lysis composition for lysing said cell or cellular compartment;
(iv) adhering said cell or cellular compartment to the surface of said microscope slide;
(v) transferring said slide with said cell or cellular compartment adhered thereto to said container thereby substantially submerging said slide in said lysis composition, wherein said slide is placed in said lysis composition at a predetermined angle with respect to the horizontal plane of between 30° and 60°, preferably about 45°, and wherein the adhered cell or cellular compartment is facing upward; and
(vi) allowing lysis of said cell or cellular compartment adhered to said slide, and removing said lysis composition from said container at a controlled and constant rate of flow by use of a liquid pump to thereby facilitate spreading of the DNA fibers on the surface of said microscope slide.

The cell is preferably a eukaryotic cell, and is preferably a mammalian cell, such as a human cell. The cell may be a tissue cell, or a blood cell, or the cell may be a cultured cell. When using cultured cells, cells are preferably about 50-70% confluent. The cellular compartment may be a mitochondrion or a nucleus, preferably an isolated nucleus.

In order to visualize newly synthesized DNA, step (i) of the method of the invention may comprise the incubation of cells with a nucleoside analog, preferably 5-ethynyl-2′-deoxyuridine (EdU). Suitable commercially available EdU may for example be provided by Jena Bioscience (CLK-001-25). Other nucleoside analogs that may be incorporated in DNA as marker include BrdU, IdU, CldU, or 5-vinyl-2′-deoxyuridine (5-VdU, e.g. Jena Bioscience CLK-050-10). The concentration of nucleoside analog may be in the range of 0.1-1000 μM. Preferably, the concentration of nucleoside analog is in the range of 1-100 μM; more preferably in the range of 5-20 μM, for example 10 μM. The nucleoside analog is suitably incubated with said cells for a period of 1-120 minutes, preferably 5-60 minutes; more preferably 10-30 minutes; even more preferably 15-25 minutes, for example 20 minutes. Following incubation with nucleoside analog, cells are preferably washed. An exemplary washing composition is phosphate-buffered saline (PBS).

Step (i) of the method of the invention may further comprises or may be preceded by a step wherein the cells are incubated with a protease that allows for the separation of cells or isolation of cells from a tissue, to thereby provide a suspension of cells. Exemplary proteases that may be used, are, e.g., trypsin, a collagenase, a dispase, or a combination thereof, preferably trypsin. The proteases may for example be provided in the form wherein the protease is dissolved in PBS, preferably PBS supplemented with Mg²⁺ and/or Ca²⁺. A suitable concentration of protease(s) is approximately 2 mg/ml. Preferably, incubation of proteases with cells is performed for 0.5-5 minutes, more preferably 1-2 minutes, and preferably at a temperature of around 37° C. Protease treatment is preferably continued until cells are round and start to detach from the cultivation plate when cultivated cells are used. After incubation, the action of the protease enzyme may be stopped by the addition of excess liquid to dilute the protease and/or by the addition of components that quench the enzymatic reaction, such as fetal calf serum (FCS).

A method of the invention may further comprise the temporary storage of cells prior to further processing, preferably at temperatures below 10° C., more preferably below 5° C., for example by placing the cells on ice.

A method of the invention may further comprise washing of the cells, preferably in cold PBS. Preferably, said PBS has a temperature below 10° C., more preferably below 5° C. Washing may be repeated one or more times.

The processing of cells further preferably comprises the step of isolating cell compartments from the cells, in particular nuclei in order to provide for isolated nuclei that are subsequently adhered to the slide and subjected to lysis and DNA/chromatin stretching. To this end, cells are preferably (re)suspended in a hypotonic buffer composition for solubilization of the cytoplasmic membrane and cytoplasmic components. The ionic salt concentration of a suitable hypotonic buffer composition may be adapted to the tonicity of the cytoplasm of the cell type that is to be analyzed. The ionic salt concentration is preferably less than 100 mM, more preferably in the range of 5-100 mM; even more preferably in the range of 5-50 mM; most preferably in the range of 5-25 mM, for example 10 mM. The ionic salt is preferably a monovalent salt, such as NaCl and/or KCl, most preferably KCl.

A hypotonic buffer composition in accordance with the invention further preferably comprises a non-ionic detergent. A very suitable non-ionic detergent is DTT. DTT keeps proteins in reduced state thereby supporting chromatin solubilization, and mediates stabilization of proteins by preventing oxidation. A very suitable hypotonic buffer composition for solubilization of cytoplasmic membrane and cytoplasm for the purpose of isolation of intact nuclei comprises 10 mM Hepes (pH 7.9), 10 mM KCl, 1.5 mM MgCl₂, 0.34 M sucrose, 10 vol % glycerol, 1 mM of DTT, a standard concentration of a protease inhibitor (e.g., cOmplete™, Mini, EDTA-free Protease Inhibitor Cocktail, Roche, #04693159001), and H₂O as the liquid carrier and/or solvent.

Following the resuspension of the cells in the hypotonic buffer composition, Triton-X-100 is preferably added to a final concentration of 0.1% (v/v), preferably in the presence of Mg and/or Ca ions, and the cells are gently mixed and preferably incubated on ice in the Triton X-100-supplemented hypotonic buffer composition, preferably for about 5 minutes. A suitable cell concentration for isolation of nuclei is about 10⁴-10⁶ cells/ml, such as 5·10⁴ cells/ml of the hypotonic buffer composition. Following this incubation, the cells are preferably washed in hypotonic buffer composition, and resuspended in a buffer composition comprising divalent cation chelating agents and protease inhibitor. A preferred buffer composition comprising divalent cation chelating agents comprises EDTA, preferably at about 3 mM, EGTA, preferably at about 0.2 mM, a reducing agent, preferably DTT, preferably at about 1 mM, and a protease inhibitor.

The isolated nuclei are now ready to be deposited on a slide.

The slide surface to which the cell or cell compartment is adhered may suitably be the surface of a microscope slide, more preferably a glass slide. A suitable commercially available microscope slide is a Superfrost Plus Micro Slide (VWR 48311-703, VWR International Inc.). Preferably, said microscope slide is pre-cleaned.

The step of adhering the cell or cellular compartment to the surface of a slide may suitably comprise the application of a small volume of a suspension comprising the cell or cell compartment onto the surface of the slide, preferably at an application location on said slide. A suitable volume of the suspension comprising the cells or cell compartments that may be applied is 0.1-1000 μl. Preferably, the volume is about 1-100 μl; more preferably 10-50 μl; even more preferably about 20 μl.

The step of adhering said cell or cellular compartment to the surface of a slide involves allowing said cell to adhere to said surface. Adherence may involve passive deposition of cells or cell compartments to the slide surface by gravity. The person skilled in the art is aware of conditions that facilitate said adhesion. Preferably, adhesion of said cell or cellular compartment is allowed by allowing the cells or cellular compartments to settle on the slide for a period of about 5 to 30 minutes, depending on the cell line. Use may be made of a humid chamber in order to avoid rapid drying of the applied liquid volume comprising the cell or cell compartment. Temperatures during adhesion of said cell to said surface are preferably around room temperature (20-25° C.). During adherence of the cell or cellular compartment to the slide, care is preferably taken that the liquid volume comprising the cell or cellular compartment is not fully dried. A cell or cell compartment is preferably not deposited onto a surface by accelerated sedimentation, such as by using a microscope slide centrifuge. Following adhesion of the cells or cell compartments to the slide, excess liquid may be drained by tilting the slide (e.g. at an angle of about 70° with the horizontal), and allowing excess liquid to run off.

The slide comprising the settled cells or cell compartments is then subjected to a process that causes lysis of the adhered cells or cell compartments and stretching of the DNA/chromatin fibers onto the surface of the slide. This process comprises the steps of providing a container holding an amount of a liquid lysis composition for lysing said cell or cellular compartment, and transferring the slide with the cell or cellular compartments adhered thereto to the container thereby substantially submerging the slide in the lysis composition. The container comprising the slide is then positioned such that the slide is placed in said lysis composition at a predetermined angle with respect to the horizontal plane of between 30° and 60°, preferably about 45°. The adhered cell or cellular compartments (i.e. the application location of the slide) facing upward. The next step is that lysis of the cells or cellular compartments adhered to the slide is allowed to occur, while at the same time the lysis composition is removed from the bottom of the container at a controlled and constant rate of flow by use of a liquid pump. This causes a very even spreading of the DNA fibers on the surface of the microscope slide. The process that causes lysis of the adhered cells or cell compartments and stretching of the DNA/chromatin fibers onto the surface of the slide is preferably carried out by placing the slide comprising the settled cells or cell compartments in a device according to the present invention, and by draining or removing the lysis composition from the container as described herein using a pump of the device.

The advantage of the use of a device according to the present invention is that it allows for the parallel preparation of high number of slides at once (e.g. up to 72 slides in the device as presented in the accompanying Figures).

Incubation of a surface-adhered cell with a lysis composition as described herein results in the lysis of the cells, as well as lysis of the nuclei. It was found that by using isolated nuclei and subjected those to lysis and stretching of DNA/chromatin as described herein, the majority of the nuclei adhered to a surface was successfully lysed. This high lysis efficiency resulted in a high quantity of analyzable fibers.

In the lysis and stretching step of a method of the invention, a controlled, constant rate of flow of lysis composition is established. In addition to causing controlled lysis of the cells or cell compartments, the constant flow gradually draws the meniscus of the lysis composition, that is in contact with the slide surface, over the surface of the slide. As a result, DNA/chromatin released from the cell due to lysis is spread over to the slide surface at a constant rate, resulting in spreading of the fibers in an approximately parallel, orderly fashion, while causing minimal breakage of the DNA/chromatin fibers.

The flow of said lysis composition from the container in methods of this present invention is controlled by a pump, preferably a peristaltic pump. The use of a pump causes the removal of the liquid, and the flow of the liquid meniscus to be very precisely controllable and constant. The use of a peristaltic pump may result in a pulsatile flow, wherein the interaction between the roller and the tube causes flow pulses. Although a continuous flow may be used in aspects of this invention, a pulsatile flow is also envisioned but not preferred. Dampening of the pump pulse may be accomplished by adjusting tubing inner diameter and roller speed. Good results with respect to even stretching of the DNA fibers have been obtained with a silicone tubing having an outer diameter of about 6 mm and an inner diameter of about 4 mm. A suitable pumping rate for removing the lysis composition from the container holding the slide(s) is about 1-100 ml/minute; more preferably about 5-20 ml/minute; even more preferably about 10 ml/minute, and preferably constant. The removal of lysis composition from the container in methods of this present invention is preferably continued until the level of the lysis composition is below the application location of the slide. It is important that the lysis or spreading composition is not removed from the container at an uncontrolled or poorly controlled rate, such as by gravitational drainage of the lysis composition from the container through a perforation in the container wall, or by pulling the slide out of the lysis composition. By use of a method or device according to the present invention, it is possible to precisely control the experimental conditions by which the cell samples are processed. This provides results that are highly reproducible between samples.

Following removal of the lysis composition from the container as described herein, the slide surface is left to dry to the air, preferably at approximately room temperature.

Following the drying of the stretched DNA/chromatin fibers, the fibers are fixed by use of a crosslinking agent comprised in a fixative composition. As an example, fixation may very suitably occur by the use of a sugar-fixative composition as described herein, for instance comprising sucrose and formaldehyde.

Fixation may be achieved by gently flowing a fixative composition over the stretched DNA/chromatin fibers in the direction of the stretching. As an example, a fixative composition may be applied to the top edge of the slide surface, which is preferably held in a tilted position and allowing the fixative composition to flow from the top edge along the surface. Preferably, the fixative composition is not applied directly onto the stretched DNA/chromatin fibers.

The stretched DNA/chromatin fibers may then be washed with alcohol, preferably ethanol, for example 95% v/v ethanol in water, preferably at a temperature of about −20° C. The alcohol may then be left to evaporate. Preferably, evaporation takes 10-300 seconds; more preferably 30-200 seconds; even more preferably 60-180 seconds; still more preferably 90-150 seconds; most preferably approximately 120 seconds. Preferably, most ethanol is evaporated after said stage of evaporation, but the surface is not completely dry.

A method of the invention preferably does not involve the use of liquid nitrogen or other liquefied gases, for fixation purposes or otherwise used in prior art protocol.

A method of the invention may further comprise a step of labeling or staining of the DNA of the stretched DNA/chromatin fibers and/or proteins associated therewith for imaging purposes. The person skilled in the art knows how to label surface-bound nucleic acids and/or associated proteins.

Labeling or staining of the stretched DNA/chromatin fibers may involve the attachment or coupling of a label for imaging purpose to nucleoside analogs incorporated into the nucleic acid or DNA to be analyzed during cultivation of the cell to be analyzed and prior to the cell lysis and chromatin stretching. The person skilled in the art knows how to attach a detectable label to a nucleoside analog. For example, the Click-iT Imaging Kit, commercially available and manufactured by Invitrogen, may be used for said purposes, whereby for example an Alexa Fluor 488 or Alexa Fluor 594 may be attached to said nucleoside analog. stretched DNA/chromatin fibers may also be labeled or stained by nuclear dyes or intercalating dyes, such as DAPI, or by Fluorescence In Situ Hybridization (FISH). Staining of proteins associated with nucleic acid or DNA to be analyzed may for instance be achieved by use of specific antibodies, such as fluorescently labeled antibodies, that bind specifically to said proteins. In fact, any nucleic acid and or protein labelling or staining method is envisioned in combination with aspects of this invention.

Slides with surface-bound nucleic acids prepared in accordance with the present invention may be directly used for imaging such as by fluorescence microscopy and/or super-resolution microscopy.

### Compositions

### Lysis Composition

The present invention provides a lysis composition that allows for efficient lysis of cells or cell compartments, and functions as spreading composition. The lysis of the cells should be efficient such that a large proportion of cells and/or nuclei is lysed. At the same time, the lysis composition should not negatively affect the nucleic acids and proteins as they are released from the cells or nuclei. Preferably, nucleic acids and proteins stay intact and associated, yet the lysis composition should cause separation of individual DNA fibers on the slide surface. The lysis composition of the invention is preferably an aqueous composition.

The lysis composition of the present invention is preferably used as a lysis and spreading composition for spreading DNA fibers from slide-adhered nuclei that have been isolated from cells. Hence, a preferred lysis composition is a lysis composition for lysing isolated nuclei. However, a lysis composition of the invention may also be used for spreading DNA fibers from slide-adhered cells.

The lysis composition of the present invention comprises MES as a buffering agent, preferably at a concentration of about 25-100 mM, and preferably at a pH of about 5.5-6.9, preferably pH 6.0-6.5.

The lysis composition of the present invention comprises a nonionic detergent. A suitable nonionic detergent may be selected from Triton X-100 (a.k.a. polyethylene glycol mono(p-(1,1,3,3-tetramethyl-butyl)phenyl)ether), Triton X-114, Nonidet P-40, Tween 80, octyl beta glucoside, MEGA 8, MEGA 9, MEGA 10, Big CHAP, deoxy Big CHAP, Brij 35 and Brij 58. Preferably, the nonionic detergent is Triton X-100. The nonionic detergent may suitably be present in the lysis composition of the invention in a concentration of about 1-3% v/v. Preferably Triton X-100 is present at about 2% v/v.

The lysis composition of the present invention comprises a metal chelator, more preferably a mixture of metal chelators to chelate a broad range of metal ions, preferably Ca++ and Mg++. Metal chelation reduces protease activity, and it was found that Mg++ chelation improves spreading of the chromatin in aspects of this invention. The metal chelator(s) may for example be selected from ethylenediaminetetraacetic acid (EDTA), ethylene glycol-bis(β-aminoethyl ether)-N,N,N′,N′-tetraacetic acid (EGTA), dimercaptosuccinic acid (DMSA), 2,3-dimercapto-1-propanesulfonix acid (DMPS), dimercaprol (BAL), deferasirox, deferiprone, deferoxamine, pentetate calcium trisodium (Ca-DTPA), pentetate zinc trisodium (Zn-DTPA), trientine, tetrathiomolybdate, dexrazoxane, N-acetyl-cysteine (NAC). Preferably, the lysis composition comprises a combination of EDTA and EGTA. The metal chelator may suitably be present in the lysis composition of the invention in a concentration of about 0.2-2 mM, preferably about 1 mM.

The lysis composition of the present invention comprises a reducing agent, preferably DTT. The reducing agent may be present in a lysis composition of the invention in a concentration of about 0.1-10 mM, more preferably 0.5-2 mM. The lysis composition of the present invention preferably comprises DTT in an amount of 1 mM.

A highly beneficial lysis composition for use in preferred aspects of this invention is an aqueous buffer comprising MES at a pH in the range of 6.0-6.5, preferably at 50 mM; and a nonionic detergent, preferably Triton X-100, preferably at 2 wt. %; the lysis composition further comprising one or more chelating agents, preferably EDTA and EGTA, preferably at 0.1 mM each, and/or a reducing agent, preferably DTT, preferably at 1 mM. This hypotonic lysis composition was found to be very suitable for stretching DNA/chromatin fibers from isolated nuclei adhered to a glass slide as described herein.

The lysis composition may further optionally comprise an ionic salt, preferably NaCl, preferably at 100 mM. The lysis composition may further optionally comprise urea, preferably at 500 mM.

In preferred embodiments, the lysis buffer of the invention does not comprise one or more of dishwasher soap, ethanol, lauramine oxide, methylisothiazolinone, phenoxyethanol, Ppg-26, sodium hydroxide, sodium laureth sulfate, triclosan and/or cyclohex-1,2-ylenediamine, preferably none of these. A lysis composition of the invention, in some preferred embodiments of aspects of this invention, may comprise a protease inhibitor, preferably in the form of a cocktail of different protease inhibitors to inhibit proteolytic activity of a broad range of proteases, and prevent degradation of proteins. The protease inhibitor(s) may for example be selected from aspartic protease inhibitors, cysteine protease inhibitors, metalloprotease inhibitors, serine protease inhibitors, threonine protease inhibitors, trypsin inhibitors. Preferably, a combination of protease inhibitors is used for the inhibition of serine, cysteine, and metalloproteases. The protease inhibitor(s) may be present in a lysis composition of the invention in a total concentration in the range 0.2-2000 μg/ml, more preferably in the range of 2-200 μg/ml.

### Fixative Composition

In some embodiments of a method of the invention, a fixative composition is used. A fixative composition for use in aspects of the present invention preferably comprises at least one fixative in an aqueous liquid. In a preferred embodiment, the fixative is formaldehyde, preferably in a concentration of about 0.5% v/v in PBS. An exemplary PBS formulation may comprise 137 mM NaCl, 2.78 mM KCl, 10 mM Na₂HPO₄ and 1.8 KH₂PO₄ in water. The fixative composition may comprise a nonionic detergent. Non-limiting examples of nonionic detergents are Triton X-100 (also referred to as polyethylene glycol mono(p-(1,1,3,3-tetramethyl-butyl)phenyl)ether), Triton X-114, Nonidet P-40 substitute, Tween 80 (also referred to as polyoxyethylene (20) sorbitan monooleate), octyl beta glucoside, MEGA 8, MEGA 9, MEGA 10, Big CHAP, deoxy Big CHAP, Brij 35 and Brij 58. Preferably, Triton X-100 is used, preferably in an amount of about 0.1% v/v. It is preferred that a formaldehyde-based fixative composition is freshly prepared prior to use and replaced after maximally 24 hours.

### Sugar-Fixative Composition

In some embodiments of a method of the invention, a sugar-fixative composition is used. A sugar-fixative composition for use in aspects of the present invention comprises a sugar and formaldehyde in water. The sugar is preferably sucrose. The amount of sucrose is preferably about 1 M. The amount of formaldehyde is preferably about 10% v/v.

### Device

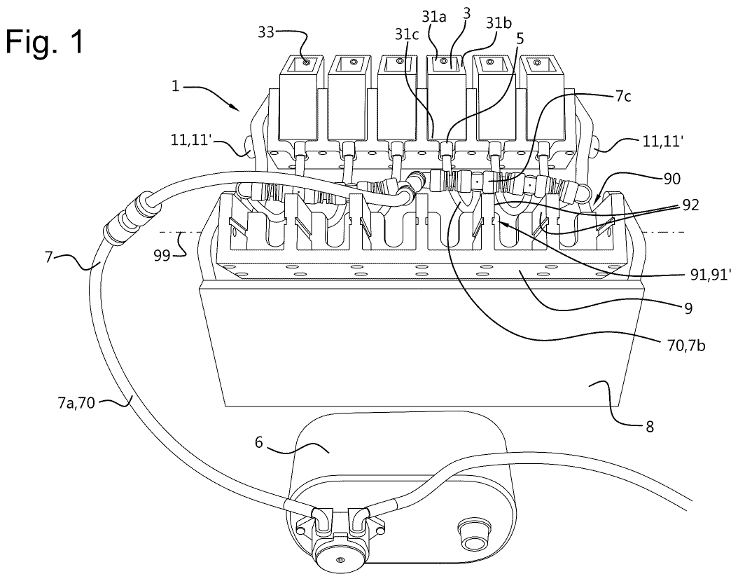

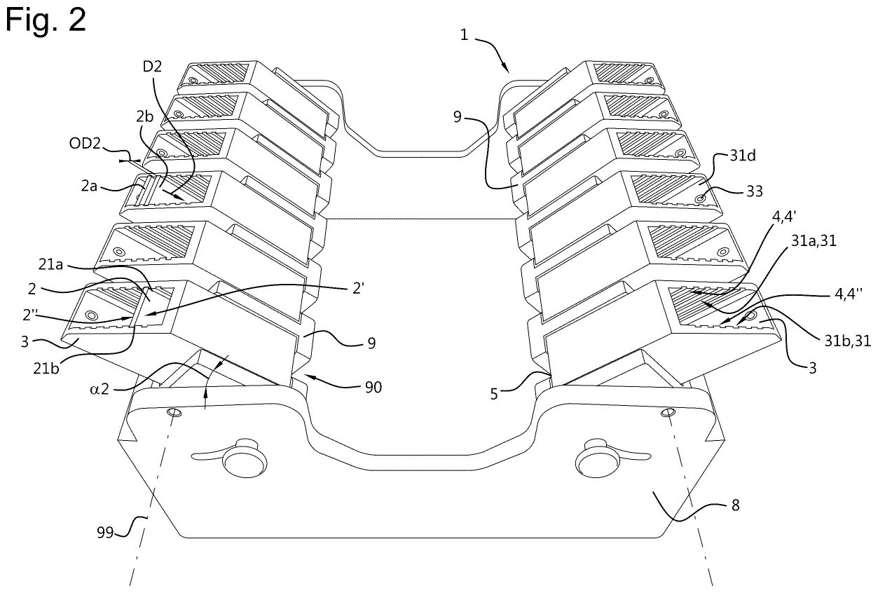

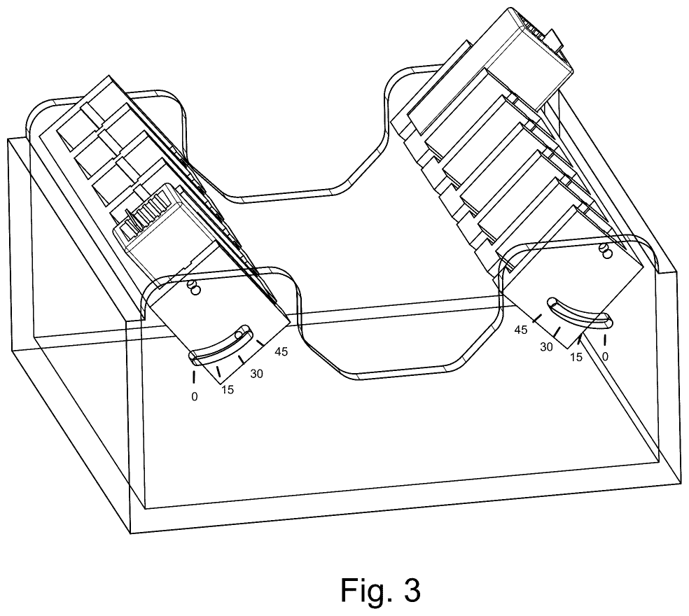

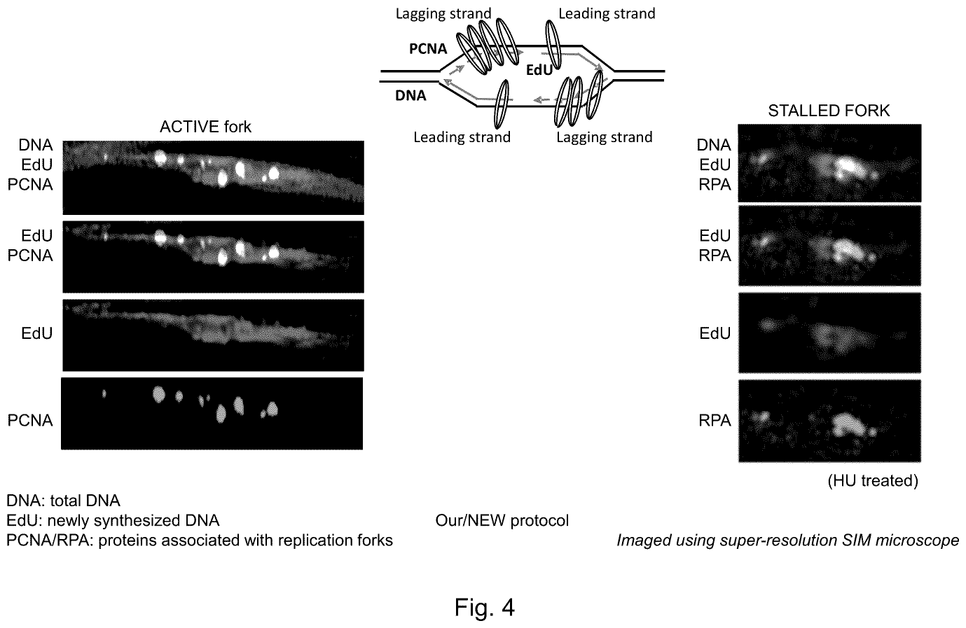

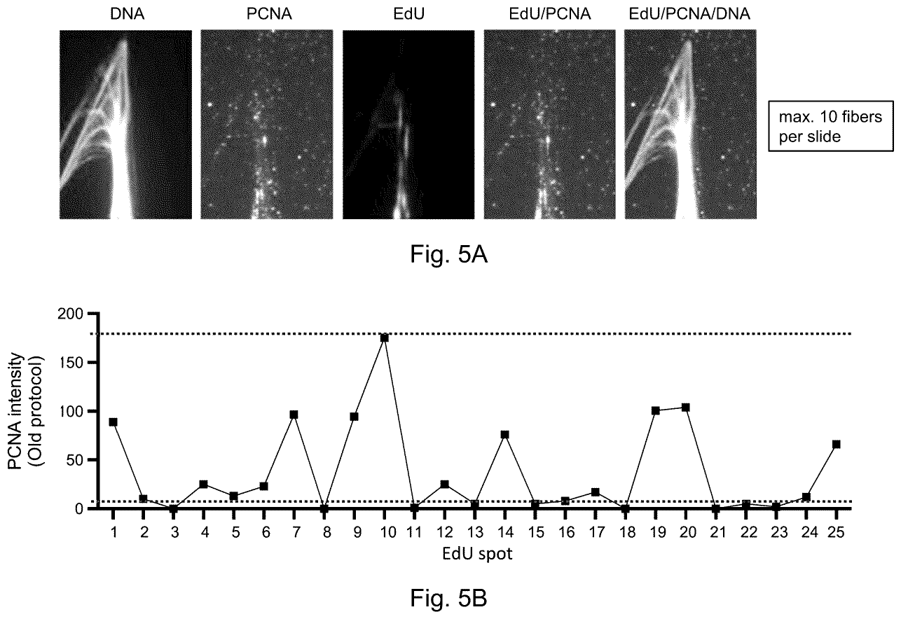

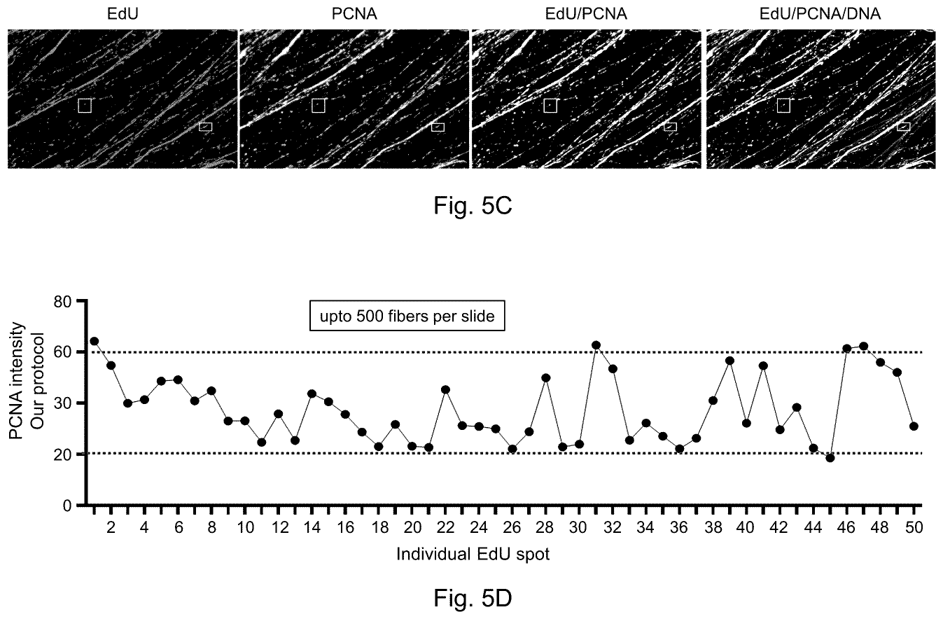

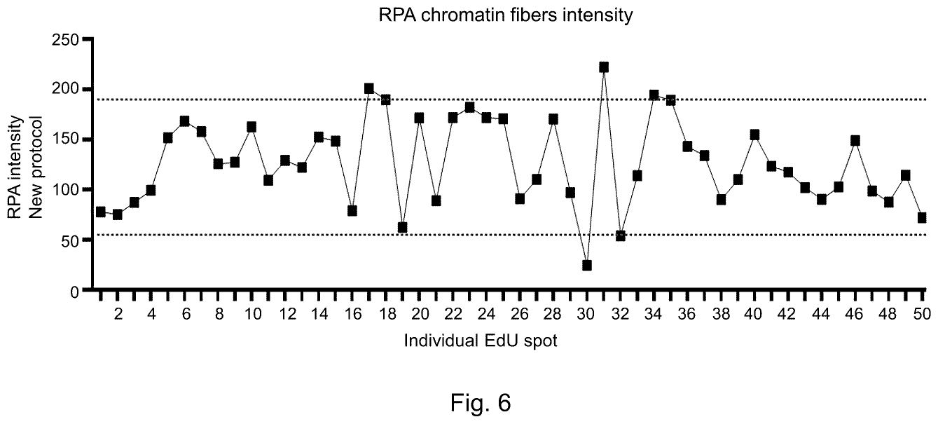

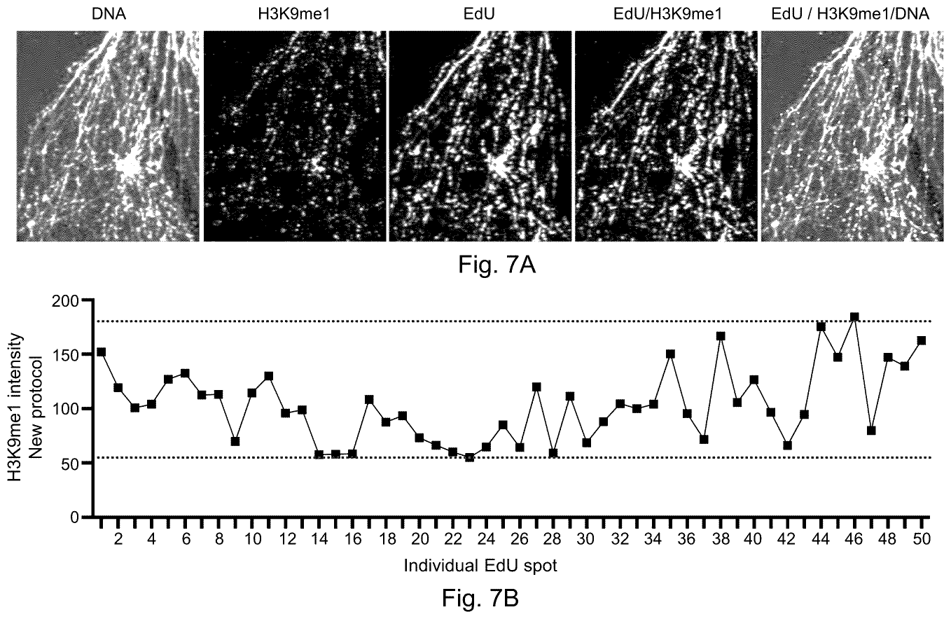

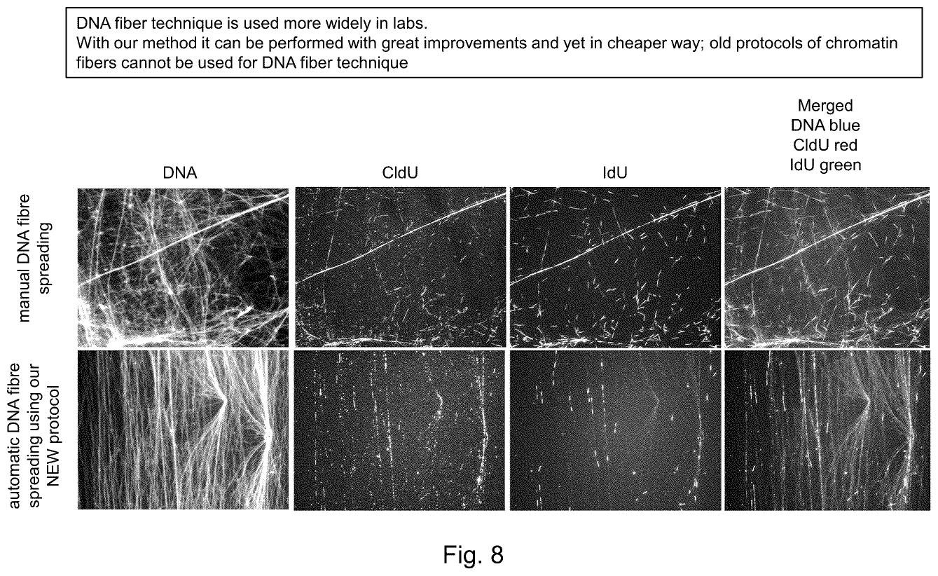

FIG. 1 shows an exemplary embodiment of a device 1 for isolating nucleic acids on a slide 2. FIG. 2 shows the same device 1.

The device 1 may for instance be arranged and/or intended for use in one of the above-mentioned methods. The device 1 comprises at least one liquid container 3, or so-called liquid chamber 3, for holding an amount of liquid, such as for instance a lysis composition.

Said liquid container 3 is arranged for holding at least one slide 2 therein in a manner in which said at least one slide 2 is substantially submerged in a liquid held in the liquid container 3 during use of the device 1. For instance, the liquid container 3, in particular side walls 31 thereof, may thereto be provided with notches 4 or slots 4 for engaging a slide 2. In particular, the liquid container 3 may comprise one or multiple sets, preferably pairs, of cooperating slots or notches 4′, 4″ in which respective side portions 21a, 21b, in particular lateral side portions, of a respective slide 2 can be received for holding said respective slide 2. A first notch 4′ of a respective set of notches may for instance be provided in a first side wall 31a of the liquid container 3, while a second notch 4″ of said respective set of notches may then be provided a second side wall 31b of the liquid container which is located opposite to the first side wall 31a. Said opposite side walls 31 may preferably extend substantially parallel with respect to each other.

In preferred embodiments, the liquid container 3 can be arranged for holding a multiple number of slides 2 therein, such as for instance at least two, at least three, or at least four, such as for instance six slides 2.

Preferably, multiple slides 2 may be held in a liquid container 3 in an interspaced manner, such that it can be facilitated that when liquid flows into or out of the liquid container 3, it may flow relatively easily between the interspaced slides.

In embodiments, the liquid container 3, in particular by means of its side walls 31, may be arranged for holding a multiple number of slides 2 therein in a manner in which multiple ones, and preferably all, of said multiple number of slides can be held substantially parallel with each other. Preferably, the device1, in particular its liquid container 3, may be arranged for holding multiple slides 2 substantially parallel to each other. As such, multiple slides 2 may be held substantially at the same angle with respect to the horizontal plane or the so-called horizontal, which may facilitate that when the container 3 is being drained, the slides may be exposed to the same conditions, in particular with respect to the flow rate. As such, as the liquid is pumped away out of the container 3, the way in which the contact between the retracting liquid and the slide is broken, can be substantially similar for the multiple slides 2, which may counteract variability and/or facilitate enabling relatively reproducible results. More preferably, the liquid container 3 may be arranged such that the slides 2 may then be substantially uniformly offset in such a manner that an offset distance OD2 between two adjacent slides 2a, 2b will be substantially equal to the offset distance between any two other adjacent slides.

It will be appreciated that, as can be seen in FIG. 2, the offset distance OD2 can be measured in a direction substantially transverse to a plane in which the respective slide 2 substantially extends.

In embodiments, the device 1 and/or the liquid container 3 may be arranged such that when the liquid container 3 is fully loaded, or at least when two adjacent slide holding locations are each holding a respective slide 2a, 2b, the offset distance OD2 between any two adjacent slides 2a, 2 may for instance at least 1 mm, preferably at least 1.5 mm, more preferably at least 2 mm, or for instance at least 3 mm, preferably 4-12 mm, still more preferably 8-12 mm. As such, it may be facilitated that the liquid can flow relatively well between two adjacent slides 2. This may counteract that when the liquid is pumped out of the liquid container 3 the flow may become relatively turbulent and/or this may facilitate a relatively laminar flow when the liquid is pumped out of the liquid container 3. As a result, nucleic acids which may be stretched out on the slide 2 by the liquid flowing along the slide 2,may be stretched out in a relatively straight manner, in particular substantially parallel with a main direction of such substantially non-turbulent flow.

On the other hand, it may also be advantageously to limit the offset distance OD2 between two adjacent slides 2a, 2b. A such, the amount of lysis buffer to be used, or other liquid to be used, can be limited. It will be appreciated that this may counteract wasting such liquid and/or may limit the costs of using the device 1. For example, the device 1 and/or the liquid container 3 may be arranged such that when the liquid container 3 is fully loaded, or at least when two adjacent slide holding locations are each holding a respective slide 2a, 2b, the offset distance OD2 between two adjacent slides 2a, 2b may be at most 15 mm, preferably at most 12 mm, more preferably about 8-12 mm, such as 10 mm.

Although in the here show exemplary embodiment, the side walls 31 of the liquid container 3 is provided with means for holding one or more slides 2, in alternative embodiments the at least one slide to be held in the liquid container 3 may be held therein in a different manner. For example, the one or multiple slides 2 may be held by means of one or multiple separate slide holders (not shown) which may be removably disposed in the liquid container 3.

In preferred embodiments, the device 1 may comprise multiple liquid containers 3, such as for instance at least two, at least three, or at least four liquid containers 3. As such slides 2 provided with different samples may be kept in separated liquid containers 3, thereby counteracting that those slides may unintentionally get mixed up. This may be advantageous, for instance as it may facilitate that nucleic acids from one or more cells of a certain first sample may be isolated in a first one of the liquid containers, whereas in a second one of the liquid containers nucleic acids from one or more cells of a certain second sample may be isolated, in particular in a manner which may counteract processing errors.

The device 1 is further arranged to hold the at least one slide 2 at a predetermined angle α2 with respect to the horizontal plane. In embodiments, said predetermined angle α2 may be between 10° and 170°, preferably between 20° and 160°, more preferably between 30° and 150°, yet more preferably between 40° and 140°. In particular, said preterminal angle α2 can be considered to be formed by the largest possible angle to be measured between the horizontal plane and any straight virtual line to be drawn on the top surface 2′ of the slide 2.

By holding the slide 2 in such an inclined position during use of the device, in particular at least when the liquid is pumped out of the liquid container 3, it can be facilitated that the liquid may flow relatively well along a top surface 2′ of the slide 2.

It is preferred that at any angle at which the slide is held, the application location of the slide faces upward with respect to the horizontal plane.

It will be appreciated that the device 1 may preferably be arranged such that the one or more slides 2 held in the liquid container 3 during use of the device 1, which one or more slides may preferably be of an elongate design and which in particular may be of a rectangular design, can extend with their longitudinal direction D2 in a substantially downward direction.

Preferably, the device 1 is arranged such that the steepest direction along the top surface 2′ of the inclined slide 2 extends substantially parallel along the longitudinal direction D2 of said slide 2 and/or along one or more side edges of said slide, in particular in case said slide would have a substantially rectangular shape. As such, when the liquid is pumped out of the liquid container 3, the liquid may flow substantially along the top surface 2′ of the slide 2 in said longitudinal direction D2 of the slide 2, which may facilitate that the nucleic acids to be isolated may become extended substantially parallel to said longitudinal direction D2 of the slide 2.

As can be seen in FIG. 1, the device 1 further comprises a pump 6 for pumping the liquid out of the liquid container 3. Preferably, the liquid container 3 may be provided with at least one liquid outlet port 5 or so-called outlets 5, which preferably may be provided at a bottom end of the container 3, such as for instance in a bottom wall 3c of the container 3. Via the liquid outlet port 5, the interior space of the liquid container 3 can be connected to the pump 6, for instance via a drainage canal 7.

In embodiments, the device 1 may comprise a drainage canal 7 provided between the liquid container 3 and the pump 6. Said drainage canal 7 may be formed at least partly by one or more tubes 70, preferably one or more flexible tubes. As is the case in the exemplary embodiment shown in FIG. 1, in embodiments, the multiple liquid containers 3 may during use be in fluid connection with the same pump 6. For instance, multiple dedicated drainage canals 7b, each of which is dedicated to one of multiple liquid containers 3, can be in fluid connection to a main drainage canal 7a or so-called master channel 7a, in particular by means of a manifold 7c.

For instance in such embodiments, one pump 6 can be used to drain multiple liquid containers 3 substantially at the same time.

In alternative embodiments, the outlets 5 of multiple liquid containers 3 may each be connected to a respective pump, such that each container 3 may be drained independently of other ones. In a preferred embodiment, multiple liquid containers 3 are each provided with a separate drainage canal, in particular one formed by a flexible tube, and said multiple drainage canals are then connected to a so-called multichannel pump, in particular a multichannel peristaltic pump, which can control the flow within the multiple drainage canals, preferably independently from each other. As such, multiple liquid containers 3 can be drained substantially simultaneously, but it may also be facilitated that the flow within different drainage canals can be varied if desired so. Besides, using a multichannel pump may for instance also enable that the liquid drained from multiple liquid containers 3 can be collected independently, which for instance may facilitate reusing the drained liquid.

In advantageous embodiments, the pump 6 may be formed by a peristaltic pump. In such embodiments, it can for instance be facilitated relatively well that the liquid container 3 can be drained at a relatively constant flow rate, which for example may facilitate that nucleic acids may be stretched out on the slide 2 relatively neatly.

Additionally or alternatively, the device 1, and in particular its pump 6, may be arranged for pumping the liquid out of the liquid container 3 at a substantially constant flow rate, for instance by providing the pump 6 in the form of a peristaltic pump. Advantageously, the device 1 and/or the pump 6 may be arranged to drain a respective liquid container 3 at a flow rate between 1 ml/min and 100 ml/min. Preferably, said flow rate may be between 2 ml/min and 50 ml/min, more preferably between 5 ml/min and 25 ml/min, such as for instance a flow rate of about 10 ml/min or about 15 ml/min.

Additionally or alternatively, the device 1, and in particular its pump 6, may be arranged for pumping the liquid out of the liquid container 3 within a certain predetermined timespan, preferably while draining it at a substantially constant flow rate. Preferably, the device 1 and/or the pump 6 may be arranged to drain a respective liquid container 3, from a full state in which one or more slides 2 held therein are substantially submerged in liquid held in said container 3 to a drained stage in which said liquid has been pumped out of it, during a period of 1 to 15 minutes, preferably a period of 2 to 10 minutes, more preferably a period of 2 to 6 minutes, such as for instance about 3 or about 4 minutes.

For example, for instance in embodiments in which one or more typical microscopes slides having a length of about 75 mm or about 3 inches are used and in which the vertical height of the volume of liquid held in the container 3 in order to keep said one or more slides substantially submerged in an inclined state of said one or more slides 2 may for instance be about 5 cm or for instance between about 4 cm and about 8 cm, the device 1 may be arranged for draining the liquid container 3 at a speed of for instance about 10-30 mm/min, seen in the vertical direction, for instance such that the container 3 may be completely drained in about 1-10 minutes, preferably 2-5 minutes, such as for instance in about 3 or about 4 minutes.

Additionally or alternatively, in embodiments, a liquid container 3, preferably one arranged for holding about six microscope slides 2, may have a liquid containing volume of for instance about 10 to 200 ml, preferably about 20 to 100 ml, such for instance about 30 ml, about 40 ml or about 50 ml. For example in such embodiments, the container 3 may during use be drained at a flow rate of between 5 ml/min and 25 ml/min, such as for instance a flow rate of about 10 ml/min or about 15 ml/min, and the device 1 may be arranged therefor.

As can be seen relatively well in FIG. 2, the device 1 may, in preferred embodiments, comprise a base portion 8 and at least one container holder 9 for retaining one or multiple liquid containers 3 therein. Preferably, the container holder 9 may comprise one or multiple container receiving compartments 90 in which at least a portion of liquid container 3 can be removably received. Preferably, the container holder 9, in particular its respective receiving compartment 90, may be provided with means for holding the container 3 a predetermined position within the container holder 9. In embodiments, the device 1 may be arranged to facilitate a form fit between the liquid container 3 and the container holder 9, thereby facilitating that the container 3 will be received by the container holder 9 in a predetermined orientation with respect to said container holder 9.

The container holder 9, in particular its respective receiving compartment 90 may thereto for instance be provided with guiding means 91 and/or restraining means 91, for instance in the form of notches 91′ or slots 91′, and/or for instance in the form of ridges or other protrusions (not shown), which may be provided in or on walls of the container holder 9, in particular walls of its respective receiving compartment 90, which for instance may be arranged to cooperate during use with corresponding means provided at the outside of the liquid container to be held, for instance means in the form of ridges or other protrusions, and/or for instance in the form of notches or slots, on or in outer walls of the container 3. This can for instance be seen relatively well in FIG. 1, in which two opposing side walls 92 of the receiving compartment 90 are provided with two guiding slots 91′ in which protrusions provided at the outer side of the liquid container 3 can be guided such as to facilitate holding said liquid container 3 a predetermined position with respect to the container holder 9.

Advantageously, the device 1 may comprise multiple container holders 9, preferably similar of even substantially identical container holders 9, each of which may be arranged for retaining at least one and preferably multiple liquid containers 3 therein.

It is noted that in embodiments, a single container holder 9 may preferably thus hold multiple liquid containers 3, preferably each held at least partly within a respective container receiving compartment 90. The container holder 9 may preferably be arranged such that multiple liquid containers 3 can be held substantially parallel to each other. This can be advantageous, as this may enable holding multiple slides 2 substantially at the same angle α2 with respect to the horizontal plane, which may facilitate counteracting variability between different samples processed by means of the device 1.

In embodiments, the angle α2 of at least one slide 2 held in the liquid container 3 can be adjusted with respect to the horizontal. In preferred embodiments, the device 1 can be arranged to adjust the orientation of the liquid container 3, in which the slide 2 is to be held, with respect to the horizontal. More preferably, the at least one container holder 9, if any, can be adjustably mounted to the base portion 8 such that during use of the device 1 the angle α2 of at least one slide 2 held in the liquid container 3 which in turn is held in the container holder 9 can be adjusted, by adjusting the inclination of the container holder with respect to the base portion 8. The adjustment of the angle is preferably continuous, and is preferably adjustable between 0° and 90° with the vertical, more preferably between 0° and 60°, most preferably between 0° and 45° with the vertical.

In embodiments, such as for instance is also the case in the here shown exemplary embodiment as can be observed in FIG. 2, inclination of the container holder 9 may be adjustable in a continuous manner. For example, as is the case in the here shown embodiment, the container holder 9 may be pivotably mounted in the base portion 8, such that said container holder 9 can be pivoted about a pivot axis 99. The device 1 may further comprise one or more locks 11, for locking the container holder 9 in the desired rotational position. Here, the lock is formed by means of screw knobs 11′. However, in alternative embodiments, said lock 11 may be formed by any other suitable means.

In some embodiments, the device 1 may be arranged such that the inclination of the slide 2 held in the container 3 can be adjusted with respect to the container holder 9. Preferably, the angle α2 between the slide 2 and the horizontal may be adjustable in a range expanding over at least 10°, preferably over at least 20°, such as over at least 30°, or over at least 40°.

In embodiments, in the steepest (or most inclined) position of a range in which the inclined position of slide 2 can be adjustable, preferably adjustable by means of a pivotable or alternatively adjustable container holder 9, the angle α2 between the slide 2 and the horizontal may be for example at least 65°, for example at least 70°, for example at least 75° or for example at least 80°. Additionally or alternatively, in the least steep position of a range in which the inclined position of slide 2 can be adjustable, preferably adjustable by means of a pivotable or alternatively adjustable container holder 9, the angle α2 between the slide 2 and the horizontal may be for example at least 10°, for example at least 15°, for example at least 20° or for example at least 25°.

Preferably the angle of the slide when positioned in the container and/or adjusted by the container holder is between 30° and 60° with the horizontal plane, more preferably between 40° and 50°, most preferably about 45°.

Preferably, the surface of the slide containing the application location for carrying the sample to be examined (top side of the slide) is upward when placed in the container, rather than downward.

Furthermore, it is noted that the device 1 may be provided with supply means (not shown) such as one or more supply canals for supplying a liquid, in particular for instance a lysis composition, in order to fill the interior of the liquid container 3 with liquid to at least a certain degree. Additionally or alternatively, the liquid container 3 may be provided with an inlet opening 33 for allowing liquid to flow into the container 3.

In embodiments, the inlet opening 33 may be provided at or near an upper end region of the container 3. This may for example facilitate that the liquid can be introduced into the liquid container 3 at a relatively low pressure, thereby for instance counteracting that that the liquid being supplied into the container 3 would unintentionally wash away material provided on the upper surface 2′ of the slide 2.

Additionally or alternatively, said inlet opening 33 may be provided in a wall 31d facing a bottom side 2″ or so-called bottom surface 2″ of a slide 2 held in the container 3, in particular facing the bottom side 2″ of the most rear or undermost slide of a series of slides 2 held in the container 3, which can counteract that the liquid being supplied into the container 3 would unintentionally wash away material provided on the upper surface 2′ of the slide 2.

It is noted that for the purpose of clarity and a concise description, features are described herein as part of the same or separate embodiments, however, it will be appreciated that the scope of the invention may include embodiments having combinations of all or some of the features described.

Further, it is noted that the invention is not restricted to the embodiments described herein. It will be understood that many variants are possible.

For example, the drainage canal 7 or a respective portion thereof, which may be formed at least partly by one or multiple flexible tubes 70, may be removably attached to the respective liquid container 3. However, in alternative embodiments, the drainage canal 7 may for example be more or less permanently fixed to a container holder 9, if any, and the device 1 may then for instance be arranged such that upon placement of the liquid container 3 into the container holder 9, a fluid connection between the liquid outlet port 5 of the container 3 and an inlet of the drainage canal 7 can be established. In such embodiments, the outlet port 5 may for instance be provided with a valve (not shown), which can be opened, preferably opened automatically upon establishing a connection between the outlet port 5 and the drainage canal 7, for instance by having said valve being pushed away towards an open position by means of a protrusion provided at or near the inlet of the drainage canal.

Additionally or alternatively, a supply canal may be removably attached to the respective liquid container 3. However, in alternative embodiments, the supply canal may for example be more or less permanently fixed to a container holder 9, if any.

Such and other variants will be apparent for the person skilled in the art and are considered to lie within the scope of the invention as formulated in the following claims.

### EXAMPLES

### Example 1. Nascent Chromatin Fiber Extraction

This procedure provides for a 50-100 fold higher efficiency of isolating intact chromatin fibers, and provides for an increased throughput when compared to other methods. The procedure thereby allows for the design of more complex experiments.

### Nascent Chromatin Labelling

The procedure was started with cultivated cells that were around 50-70% confluent. EdU was added to a final concentration of 10 μM and incubated for 20 minutes. The labelled cells were then washed with 10 mL PBS. An amount of 1.5 ml of trypsin was added and cells were incubated at 37° until cells were round and started to detach from the plate. An amount of 1.5 ml of warm media was added in order to saturate the trypsin and stop the enzymatic reaction. The cells were then collected and placed immediately on ice. Cells were kept on ice between each step unless otherwise indicated. Cells were spun down at 400×g for 5 minutes at 4° C., medium was removed and cells were resuspended in 1 ml of cold PBS. Cells were transferred to a 1.5 ml Eppendorf tube and spun at 400×g for 5 minutes at 4° C. The supernatant was carefully removed and cells were resuspended in 250 μL of cold PBS.

### Isolation of Cell Nuclei Prior to Chromatin Fiber Extraction

This key step in the production of high-quality fibers is totally different from pre-existing procedures and represent a major improvement toward the reproducible observation of high quality chromatin fibers.

Following the preparation of the cells as described above, the cells were subjected to a procedure for solubilization of the cell cytoplasm and isolation of nuclei. To this end, cells were resuspended in 1 ml of a buffer A (e.g. 1 ml) comprising 10 mM Hepes (pH 7.9), 10 mM KCl, 1.5 mM MgCl₂, 0.34 M sucrose, 10 vol % glycerol, 1 mM of DTT, 1× protease inhibitor (cOmplete™, Mini), in H₂O. An amount of 0.1% (v/v) of Triton-X-100 was added, and mixed gently with the cells by rotating the tube 5 times. The mixture was allowed to incubate on ice for 5 minutes. The mixture was then centrifuged for 4 minutes at 1300×g, at 4° C. The supernatant was carefully removed, and the pellet was gently resuspended in 1 ml buffer A. The mixture was then centrifuged for 4 minutes at 1300×g, at 4° C. The supernatant was carefully removed, and the pellet was gently resuspended in 1ml of buffer C (3 mM EDTA, 0.2 mM EGTA, 1 mM DTT, 1× protease inhibitor, in H₂O). EDTA and EGTA as chelating agents were used to remove divalent cations (Ca++ and Mg++) to inhibit the action of proteases and facilitated IF quality due to intact proteins on isolated fibers. The isolated nuclei were then ready to be deposited on a slide.

### Slide Adherence of Nuclei and On-Slide Chromatin Extraction and Fixation

A slide was placed into a humid chamber and a 20 μl drop of nuclei suspension was placed onto the slide. The humid chamber was closed and the cells were allowed to settle on the slide for 5 to 30 minutes (depending on the cell line). The slide was removed from the humid chamber and tilted at an angle of 70°. The cells that had settled onto the slide stuck to the glass, while the liquid was allowed to flow down the slide. After 2 minutes, the slide was transferred to a container of the device described herein and presented in the accompanying Figures, the container containing 40 ml of buffer D (50 mM MES (pH 6.5), 0.1 mM EDTA, 0.1 mM EGTA, 1 mM DTT, 2 wt. % Triton X-100, in H₂O). The slides were incubated in buffer D for 10 to 30 minutes. The slides were positioned at an angle of 45° with the horizontal plane. Following the incubation, the flow valve was opened and buffer D was pumped out of the container at a constant rate of 15 ml/min with a peristaltic pump (GROTHEN G628-1 (4 to 18 ml/min) peristaltic pump, silicone tubing with an outer diameter of 6 mm and an inner diameter of 4 mm).

It was found that this step represented a major improvement in the nascent chromatin fiber stretching. While prior art protocols relied on manual pulling of the slide from the buffer, or on a non-controlled flow resulting from draining of the fluid through holes pierced at the bottom of the container, in order to stretch the chromatin fibers, it was found that these prior art procedures made it almost impossible to provide reproducible results and provide identical experimental conditions from sample to sample. In the present procedure, the device allows for the preparation of slides in a systematic way, which also save time and labor. The control of the flow of the spreading composition allows the higher output of homogeneous and reproducible spreading of the chromatin fibers.

Once the buffer D was pumped out, the remaining lysis buffer was allowed to evaporate to air for a maximum of 5 minutes. An amount of 20 μL of freshly prepared 1M sucrose/10% formaldehyde solution was placed on the top edge of the slide above the fibers and was allowed to flow over the sample. Care was taken that the solution was not placed directly on the chromatin. A cover slip was added and the slide was incubated with the sucrose/formaldehyde solution for 2 min. Excess fixative was removed, and the slides were transferred to a coplin jar containing 95% ethanol that was pre-chilled to −20° C. and incubated at −20° C. for 10 minutes. The slides were removed from the ethanol and the ethanol was allowed to evaporate for 2 min (until most was evaporated but slide was not completely dry). An amount of 1 ml of freshly prepared fixative solution was added to the slide and incubated for 5 minutes at room temperature. The indicated fixation steps, although similar to prior art methods, was optimized and improved efficiency. In particular relative to liquid nitrogen-mediated flash freezing were found to be an additional hurdle and to add to the cost of the method. The present procedure uses no liquid nitrogen fixation steps. This omission was found not to impair the quality of the fibers while allowing for the observation of more chromatin fiber per slide.

The fixative solution was then drained from the slide, and the slide was transferred to a coplin jar containing 50 ml 1×PBS for a quick (1 minute) wash. The slide was removed from the PBS and placed at a 45° angle while allowing the PBS to drain off but not allowed to dry out. Spend PBS was discarded and the coplin jar was filled with fresh PBS and slides were reinserted for three 5 minutes washes. The slides were at that moment ready for staining of the nascent chromatin fibers (at this stage slides can be kept in PBS overnight at 4° C.).

The above-described fixation procedure provides very good results. However, using the device of the present invention, also good results were obtained using more conventional fixation methods such as incubating the slides for 15 minutes at room temperature in 4% paraformaldehyde, or incubating the slides in 100% methanol (pre-chilled) at −20° C. for 15 minutes.

### Nascent Chromatin Fiber Visualization

Slides containing the extended chromatin fibers were then stained to visualize nascent chromatin using the standard click reaction. Visualization of nascent chromatin was also combined with immuno-fluorescent labelling of proteins of interest to assess their presence/dynamics at newly replicated chromatin (nascent chromatin).

### Example 2. Stretching of DNA Fiber

It is of interest to mention that the system of the present invention can also be used to observe DNA fiber (without proteins). The DNA fiber technique is widely used in the academic field as an alternative to a highly expensive DNA combing technique (Genomic Vision). However, this technique presents a major weak point. In order for DNA stretch to be visible on a slide, cells must be mechanically lysed on a slide. This is usually done by the manual pipetting up-and-down for several time of a drop of lysis buffer containing cells followed by the tilting of the slide to allow the drop containing the DNA that have been released from the lysed cells to flow along the slide resulting in the spreading of the DNA. The problem here is that it is impossible to reproduce the exact same conditions from sample to sample resulting in variation in the quality of the fibers.

In order to overcome these problems, the present Example describes the use of the system as disclosed herein and its use as an alternative to the sub-optimal DNA fiber technique. Moreover, the system can also fully replace the DNA combing technique. A protocol for DNA fiber extension using the system of the present invention was developed as follows.

Cells were resuspended at a concentration of 2.5×10⁴ cells/ml in PBS. A 25 μL drop of cell suspension was deposited at the top of a Superfrost Plus microslides (VWR 48311-703, precleaned) and transfer to a humid chamber for 10 minutes to allow the cells to settle. The slide was removed from the humid chamber and placed in a dry air oven (hybridization oven) at 37° C. and the liquid was allowed to evaporate almost completely from the slide (the drop on the slide was not allowed to dry completely. They were kept a bit moist). The slide was inserted into a container of the device of the present invention containing 40 ml of lysis buffer (200 mM Tris-HCl pH 7.5, 50 mM EDTA, 1% SDS, in H₂O). The slide was incubated in the lysis buffer for 5 minutes. Following incubation, the flow valve was opened and the lysis buffer was pumped out of the container at a constant rate of flow of 10 ml/min with a peristaltic pump. Once the buffer had flown out, the remaining lysis buffer was allowed to evaporate completely from the slide. Once the slide was completely dry it was placed in a coplin jar containing a 3:1 mixture of methanol: acetic acid glacial, and was left to incubate for 10 minutes at room temperature or overnight at 4° C. Following this, the slides were ready for immuno-labelling of DNA fibers. This procedure could be combined with Fluorescence In Situ Hybridization (FISH) to probe for specific genomic loci to detect DNA sequences/chromosomal abnormalities.

### Example 3. Studies on Chromatin Fiber Spreading (Lysis) Buffers

A number of buffers was tested for use in aspects of the invention as described above.

Buffer 1a: Tris-base; high NaCl; low pH (old protocols recipe: overall poor for lysis, spreading, quality & quantity of fibers)

TABLE 1
Buffer 1a.

| | Stock | Final | Volume for |
|---|---|---|---|
| Component | concentration | concentration | 100 mL |
| UREA | 1M | 500 mM | 50 mL |
| NaCl | 5M | 500 mM | 10 mL |
| Tris-base pH 7.5 | 1M | 10 mM | 1 mL |
| Triton X-100 | 10% | 2% | 20 mL |
| H₂O | | | 19 mL |

### Quality Control

Lysis efficiency: −
Spreading quality: −
Immuno Fluorescence quality: −−
Quantity of analyzable fibers: −

Buffer 1b: Tris-base; Low NaCl; low pH (less salt conc. with low pH helps in IF quality)

TABLE 2
Buffer 1b.

| | Stock | Final | Volume for |
|---|---|---|---|
| Component | concentration | concentration | 100 mL |
| UREA | 1M | 500 mM | 50 mL |
| NaCl | 5M | 100 mM | 2 mL |
| Tris-base pH 7.5 | 1M | 10 mM | 1 mL |
| Triton X-100 | 10% | 2% | 20 mL |
| H₂O | | | 27 mL |

### Quality Control

Lysis efficiency: −
Spreading quality: −
Immuno Fluorescence quality: +
Quantity of analyzable fibers: −

Buffer 2a: Tris-base; High NaCl; high pH (high salt is bad for IF quality but high pH in combination helps in lysis)

TABLE 3
Buffer 2a.

| | Stock | Final | Volume for |
|---|---|---|---|
| Component | concentration | concentration | 100 mL |
| UREA | 1M | 500 mM | 50 mL |
| NaCl | 5M | 500 mM | 10 mL |
| Tris-base pH 10 | 1M | 10 mM | 1 mL |
| Triton X-100 | 10% | 2% | 20 mL |
| H₂O | | | 19 mL |

### Quality Control

Lysis efficiency: +
Spreading quality: +
Immuno Fluorescence quality: −−
Quantity of analyzable fibers: +

Buffer 2b: Tris-base; Low NaCl; high pH (high pH helps in spreading but IF quality remains poor)

TABLE 4
Buffer 2b.

| | Stock | Final | Volume for |
|---|---|---|---|
| Component | concentration | concentration | 100 mL |
| UREA | 1M | 500 mM | 50 mL |
| NaCl | 5M | 100 mM | 2 mL |
| Tris-base pH 10 | 1M | 10 mM | 1 mL |
| Triton X-100 | 10% | 2% | 20 mL |
| H₂O | | | 27 mL |

### Quality Control

Lysis efficiency: +
Spreading quality: +
Immuno Fluorescence quality: −
Quantity of analyzable fibers: +

Buffer 3: Tris-base; high NaCl; high pH; no UREA (UREA seems to be critical for lysis)

TABLE 5
Buffer 3.

| | Stock | Final | Volume for |
|---|---|---|---|
| Component | concentration | concentration | 100 mL |
| NaCl | 5M | 100 mM | 2 mL |
| Tris-base pH 10 | 1M | 10 mM | 1 mL |
| Triton X-100 | 10% | 2% | 20 mL |
| H₂O | | | 77 mL |

Note:
Urea helps in breaking the hydrogen bonds and thus, facilitates lysis of cell membrane and stretching of chromatin fibers but it also unfolds isolated chromatin fiber intact proteins, so IF quality remains poor with urea in buffer.

### Quality Control

Lysis efficiency: −
Spreading quality: −
Immuno Fluorescence quality: −
Quantity of analyzable fibers: −

Buffer 4: Tris replaced by MES (lysis good, spreading good, and thus the quantity of analyzable fibers)

TABLE 6
Buffer 4.

| | Stock | Final | Volume for |
|---|---|---|---|
| Component | concentration | concentration | 100 mL |
| UREA | 1M | 500 mM | 50 mL |
| NaCl | 5M | 100 mM | 2 mL |
| MES pH 6 | 0.5M | 50 mM | 10 mL |
| Triton X-100 | 10% | 2% | 20 mL |
| H₂O | | | 18 mL |

Note:
MES (2-(N-morpholino)ethanesulfonic acid) mimics the cell environment and therefore, optimum for biochemical experiments. It facilitates minimal salt effects, enzymatically stable, maximal water solubility and doesn't bind efficiently with cations such as Ca, Mg that can harm the proteins and DNA structure (i.e. chromatin integrity).

### Quality Control

Lysis efficiency: ++
Spreading quality: ++
Immuno Fluorescence quality: +
Quantity of analyzable fibers: ++

Buffer 5: MES, without UREA (immunofluorescence quality improves but lysis efficiency becomes poor and so the quantity of individual fibers)

TABLE 7
Buffer 5.

| | Stock | Final | Volume for |
|---|---|---|---|
| Component | concentration | concentration | 100 mL |
| NaCl | 5M | 100 mM | 2 mL |
| MES pH 6.5 | 0.5M | 50 mM | 1 mL |
| Triton X-100 | 10% | 2% | 20 mL |
| H₂O | | | 77 mL |

Note:
Urea helps in breaking the hydrogen bonds and thus, facilitates lysis of cell membrane and stretching of chromatin fibers but it also unfolds isolated chromatin fiber intact proteins, so IF quality remains poor with urea in buffer.

### Quality Control

Lysis efficiency: −
Spreading quality: ++
Immuno Fluorescence quality: ++
Quantity of analyzable fibers: −

Buffer 6: best buffer for optimal chromatin fiber spreading (adding EDTA EGTA improves immunofluorescence quality dramatically)

TABLE 8
Buffer 6.

| | Stock | Final | Volume for |
|---|---|---|---|
| Component | concentration | concentration | 100 mL |
| MES pH 6.5 | 0.5M | 50 mM | 1 mL |
| EDTA | 0.5M | 0.1 mM | 0.02 mL |
| EGTA | 0.5M | 0.1 mM | 0.02 mL |
| Triton X-100 | 10% | 2% | 20 mL |
| H₂O | | | 79 mL |

Note:
EDTA and EGTA are chelating agents to remove divalent cations (Ca++ and Mg++) to inhibit the action of proteases and thus facilitated IF quality due to intact proteins on isolated fibers.

### Quality Control

Lysis efficiency: ++
Spreading quality: ++
Immuno Fluorescence quality: +++
Quantity of analyzable fibers: ++

### Example 4. Final Procedure for Optimal Lysis and Chromatin Fiber Spreading Using Device of the Present Invention

Cells were lyses in a tube to remove cytoplasm. Nuclei were extracted from the cell and nuclei were swollen in hypotonic solution, and then resuspended in new spreading buffer.

Buffer A: solubilization of the cell cytoplasm

TABLE 9
Buffer A: solubilization of the cell cytoplasm.

| | | | Volume for |
|---|---|---|---|
| | Stock [ ] | Final [ ] | 5000 μL |
| Hepes pH 7.9 | 1M | 10 mM | 50 |
| KCl | 1M | 10 mM | 50 |
| MgCl2 | 1M | 1.5 mM | 7.5 |
| sucrose | 2M | 0.34M | 850 |
| glycerol | 100% | 10% | 500 |
| DTT | 1M | 1 mM | 5 |
| Protease inhibitor | 7x | 1x | 714.5 |
| H2O | | | 2823 |

1. Resuspend cells in buffer A (e.g. 1 mL)
2. Add 0.01 volume of buffer B (e.g. 10 μL) [buffer B: 10% Triton-X100].
3. Mix gently by rotating the tube 5 times. And incubate on ice for 5 minutes
4. Spin for 4 minutes at 1300 g, 4°
5. Carefully remove supernatant
6. Gently ressuspend the pellet in 1 ml buffer A
7. Spin for 4 minutes at 1300 g, 4° C.
8. Carefully remove supernatant
9. Gently ressuspend the pellet in 1 ml buffer C

TABLE 10
Buffer C: Nuclei resuspension buffer

| | | | Volume for |
|---|---|---|---|
| | Stock [ ] | Final [ ] | 5000 μL |
| EDTA | 500 mM | 3 mM | 30 |
| EGTA | 500 mM | 0.2 mM | 2 |
| DTT | 1M | 1 mM | 5 |
| Protease inhibitor | 7x | 1x | 714.5 |
| H2O | | | 4248.5 |

Note:
DTT is a reducing agent that helps keeping proteins in reduced state which helps in chromatin solubilization; and also mediates stabilization of proteins by protecting them from oxidation stress.

10. Nuclei are now ready to be deposited on a slide as follows:

Place a slide into a humid chamber and place a 20 μl drop of cell suspension onto the slide. Close the humid chamber and allow the cells to settle on the slide for 5 to 30 minutes (depending on the cell line).

Remove the slide from the humid chamber and tilt it at 70° angle. The cells that have settled onto the slide will stick to the glass, but the liquid will flow down the slide. After 2 minutes transfer the slide to the buffer D and place them in the device of the present invention.

Buffer D (Buffer 7): best buffer for optimal lysis and chromatin fiber spreading (UREA and Tris are omitted, but used in all previous protocols)
[new replacements: MES, EDTA, EGTA and DTT]. Furthermore, a step is added to isolate nuclei in Eppendorf tube and lysing nuclei instead of whole cell on the slide.

TABLE 11
Buffer D (Buffer 7)

| | Stock | Final | Volume for |
|---|---|---|---|
| Component | concentration | concentration | 100 mL |
| MES pH 6.5 | 0.5M | 50 mM | 1 mL |
| EDTA | 0.5M | 0.1 mM | 0.02 mL |
| EGTA | 0.5M | 0.1 mM | 0.02 mL |
| DTT | 1M | 1 mM | 0.1 mL |
| Triton X-100 | 10% | 2% | 20 mL |
| H₂O | | | 78.86 mL |

### Quality Control

Lysis efficiency: +++
Spreading quality: +++
Immuno Fluorescence quality: +++
Quantity of analyzable fibers: +++

1. A method of spreading DNA fibers on a surface of a microscope slide for microscopic analysis, the method comprising the steps of:
(i) providing a cell or cellular compartment comprising DNA fibers to be analyzed;
(ii) providing a microscope slide for microscopic analysis;
(iii) providing a container holding an amount of a liquid lysis composition for lysing said cell or cellular compartment;
(iv) adhering said cell or cellular compartment to the surface of said microscope slide;
(v) transferring said slide with said cell or cellular compartment adhered thereto to said container thereby substantially submerging said slide in said lysis composition, wherein said slide is placed in said lysis composition at a predetermined angle with respect to the horizontal plane of between 45° and 90°, and wherein the adhered cell or cellular compartment is facing upward; and
(vi) allowing lysis of said cell or cellular compartment adhered to said slide, and removing said lysis composition from said container at a controlled and constant rate of flow by use of a liquid pump to thereby facilitate spreading of the DNA fibers on the surface of said microscope slide.

2. The method according to claim 1, wherein said cellular compartment is a cell nucleus, wherein step (i) comprises the steps of providing a cell comprising DNA fibers to be analyzed, solubilizing the cell membrane and extracting the intact cell nucleus from said cell, and wherein step (iv) comprises the step of adhering said intact cell nucleus to the surface of said microscope slide.

3. The method according to claim 1, wherein said lysis composition is an aqueous buffer comprising as a buffering agent 2-(-N-morpholino)ethanesulfonic acid (MES) at a pH in the range of 6.0-6.5; and
a nonionic detergent.

4. The method according to claim 1, wherein said DNA fiber is a chromatin fiber.

5. The method according to claim 4, wherein said lysis composition is hypotonic.

6. The method according to claim 1, wherein said DNA fiber is a protein-free DNA fiber, and wherein said lysis composition comprises sodium dodecyl sulphate (SDS).

7. The method according to claim 1, wherein said staining of the thus spread DNA fibers comprises immunofluorescent staining.

8. The method according to claim 1, wherein step (i) further involves incubation with a nucleoside analog, and allowing incorporation in said nucleoside analog in said DNA fiber.

9. The method according to claim 1, wherein said microscopic analysis comprises fluorescence microscopy.

10. A hypotonic lysis composition for use in a method according to claim 1, the lysis composition consisting of
50 mM MES (pH 6.0-6.5),
0.1 mM EDTA,
0.1 mM EGTA,
1 mM DTT,
2 wt. % Triton X-100.

11. A microscope slide provided with DNA fibers spread by the method according to claim 1.

12. A device for spreading DNA fibers on a microscope slide, said device being arranged for use in a method according to claim 1, comprising a liquid container for holding an amount of liquid, wherein the liquid container is arranged for holding at least one slide therein in a manner in which said at least one slide is substantially submerged in a liquid, held in the liquid container during use of the device, wherein the device is arranged to hold the at least one slide at a predetermined angle with respect to the horizontal plane, said angle being between 10° and 170°, wherein the device further comprises a pump for pumping the liquid out of the liquid container.

13. A device according to claim 12, wherein the pump is a peristaltic pump, and/or wherein the device, and in particular the pump, is arranged for pumping the liquid out of the liquid container at a substantially constant flow rate.

14. A device according to claim 12, wherein the liquid container is arranged for holding a multiple number of slides therein.

15. A device according to claim 12, wherein the device comprises a base portion and at least one container holder for retaining the at least one liquid container therein.

16. A device according to claim 14, wherein the multiple number of slides are held in said liquid container in a removable insert comprising slits for holding individual slides at predetermined offset.

17. Use of the device according to claim 12.

18. A system for the preparation of a microscope slide comprising spread chromatin fibers on the surface of said slide for microscopic analysis, the system comprising (a) an aqueous lysis composition consisting of 50 mM MES (pH 6.5), 0.1 mM EDTA, 0.1 mM EGTA, 1 mM DTT, and 2 wt. % Triton X-100, and (b) a device according to claim 12.
